(19) United States
(12) Patent Application Publication
(10) Pub. No.: US 2005/0136477 A1
(43) Pub. Date: Jun. 23, 2005

(54) Methods for isolating nucleic acids from biological and cellular materials

(76) Inventor: Hashem Akhavan-Tafti, Howell, MI (US)

Correspondence Address: LUMIGEN, INC., 22900 W. EIGHT MILE ROAD, SOUTHFIELD, MI, 48034, US

(21) Appl. No.: 11/061,984

(22) Filed: Feb. 18, 2005

Related U.S. Application Data
(63) Continuation-in-part of application No. 10/942,491, filed on Sep. 16, 2004.
Application No. 10/942,491 is a continuation-in-part of application No. 10/714,763, filed on Nov. 17, 2003.
Application No. 10/942,491 is a continuation-in-part of application No. 10/715,284, filed on Nov. 17, 2003.
(60) Provisional application No. 60/638,621, filed on Dec. 22, 2004.

(51) Int. Cl. C12Q 1/68; C07H 21/04

(52) U.S. Cl. 435/6; 536/25.4

(57) ABSTRACT

Methods of isolating nucleic acids from samples of biological or cellular material are disclosed which use solid phase binding materials and which avoid the use of any lysis solution or coating. The use of the solid phase binding materials unexpectedly allow the nucleic acid content of cells to be freed and captured directly and in one step. The new methods represent a significant simplification over existing methods. Nucleic acids can be captured and released in a form suitable for downstream processing in under five minutes. Preferred solid phase materials for use with the methods and compositions of the invention comprise a quaternary onium nucleic acid binding portion.

### CROSS REFERENCE TO RELATED APPLICATION

The present application is a continuation-in-part of Applicants' co-pending Provisional application Ser. No. 60/638,621 filed on Dec. 22, 2004 and Applicants' co-pending application Ser. No. 10/942,491 filed on Sep. 14, 2004 which is a continuation-in-part of Applicants' co-pending U.S. application Ser. No. 10/714, 763, filed on Nov. 17, 2003 and U.S. application Ser. No. 10/715,284, filed on Nov. 17, 2003.

### FIELD OF THE INVENTION

The present invention relates to simplified methods for capturing and isolating nucleic acids, particularly total genomic nucleic acid from materials of biological origin, especially from blood and bacterial culture. The present invention further relates to kits containing solid phase binding materials useful in these methods.

### BACKGROUND OF THE INVENTION

Molecular diagnostics and modern techniques in molecular biology (including reverse transcription, cloning, restriction analysis, amplification, and sequence analysis), require the extraction of nucleic acids. Obtaining nucleic acid is complicated by the complex sample matrix in which target nucleic acids are found. Such samples include, e.g., cells from tissues, cells from bodily fluids, blood, bacterial cells in culture, agarose gels, polyacrylamide gels, or solutions resulting from amplification of target nucleic acids. Sample matrices often contain significant amounts of contaminants which must be removed from the nucleic acid(s) of interest before the nucleic acids can be used in molecular biological or diagnostic techniques.

Conventional techniques for obtaining target nucleic acids from mixtures produced from cells and tissues as described above, require the use of hazardous chemicals such as phenol, chloroform, and ethidium bromide. Phenol/chloroform extraction is used in such procedures to extract contaminants from mixtures of target nucleic acids and various contaminants. Alternatively, cesium chloride-ethidium bromide gradients are used according to methods well known in the art. See, e.g., Molecular Cloning, ed. by Sambrook et al. (1989), Cold Spring Harbor Press, pp. 1.42-1.50. The latter methods are generally followed by precipitation of the nucleic acid material remaining in the extracted aqueous phase by adding ethanol or 2-propanol to the aqueous phase to precipitate nucleic acid. The precipitate is typically removed from the solution by centrifugation, and the resulting pellet of precipitate is allowed to dry before being resuspended in water or a buffer solution for further use.

Simpler and faster methods have been developed which use various types of solid phases to separate nucleic acids from cell lysates or other mixtures of nucleic acids and contaminants. Such solid phases include chromatographic resins, polymers and silica or glass-based materials in various shapes and forms such as fibers, filters and coated containers. When in the form of small particulates, magnetic cores are sometimes provided to assist in effecting separation.

DNA Extraction from Whole Blood DNA is extracted from leucocytes in blood. Blood is typically treated to selectively lyse erythrocytes and after a precipitation or centrifugation step, the intact leucocytes are separately lysed to expose the nucleic acid content. Proteins are digested and the DNA obtained is isolated with a solid phase then used for determination of sequence polymorphism, sequence analysis, RFLP analysis, mutation detection or other types of diagnostic assay. A method disclosed in EP0796327B1 involves mixing a cell-containing sample such as a bacterial culture or whole blood and a lysis detergent in the presence of a particulate solid support. The present method in contrast omits the use of any detergent or lysis solution. Another method involves selectively capturing leucocytes from whole blood with antibody-coated particles, followed by a step of lysing the captured leucocytes and capturing the released nucleic acid on a solid support (U.S. Patent Application Publication 2003/0180754A1).

Nucleic Acid extraction from bacteria U.S. Pat. No. 5,990,301 discloses a method for isolating nucleic acids from bacteria or viruses by lysis followed by isolating the freed nucleic acids on an anion exchanger, eluting with solutions of controlled ionic strength, and then treating with a detergent or a chromatographic support to remove endotoxins. Kits containing a solid binding support material have been developed and are available commercially for use in methods of isolating genomic from bacterial culture and from whole human blood. Procedures provided by the manufacturers invariably specify that cells must be lysed before commencing with removal and purification of the nucleic acid. An additional precipitation step is sometimes also employed before use of the solid support (e.g., K. Smith, et al., J. Clin. Microbiol., 41(6), 2440-3 (2003); P. Levison, et al., J. Chromatography A, 827, 337-44 (1998)).

Solid Phase Materials One type of solid phase used in isolating nucleic acids comprises porous silica gel particles designed for use in high performance liquid chromatography (HPLC). The surface of the porous silica gel particles is functionalized with anion-exchangers to exchange with plasmid DNA under certain salt and pH conditions. See, e.g. U.S. Pat. Nos. 4,699,717, and 5,057,426. Plasmid DNA bound to these solid phase materials is eluted in an aqueous solution containing a high concentration of a salt. The nucleic acid solution eluted therefrom must be treated further to remove the salt before it can be used in downstream processes.

Other silica-based solid phase materials comprise controlled pore glass (CPG), filters embedded with silica particles, silica gel particles, diatomaceous earth, glass fibers or mixtures of the above. Each silica-based solid phase material reversibly binds nucleic acids in a sample containing nucleic acids in the presence of chaotropic agents such as sodium iodide (NaI), guanidinium thiocyanate or guanidinium chloride. See e.g. U.S. Pat. Nos. 5,234,809, 6,582,922. Such solid phases bind and retain the nucleic acid material while the solid phase is subjected to centrifugation or vacuum filtration to separate the matrix and nucleic acid material bound thereto from the remaining sample components. The nucleic acid material is then freed from the solid phase by eluting with water or a low salt elution buffer. Commercially available silica-based solid phase materials for nucleic acid isolation include, e.g., Wizard™ DNA purification systems products (Promega, Madison, Wis.), the QiaPrep DNA isolation systems (Qiagen, Santa Clarita, Calif.), High Pure (Roche), and GFX Micro Plasmid Kit, (Amersham).

Polymeric resins in the form of particles are also in widespread use for isolation and purification of nucleic acids. Carboxylate-modified polymeric particles (Bangs, Agencourt) are known. Polymers having quaternary ammonium head groups are disclosed in European Patent Application Publ. No. EP 1243649A1. The polymers are inert carrier particles having covalently attached linear non-crosslinked polymers. This type of polymeric solid phase is commonly referred to as a tentacle resin. The linear polymers incorporate quaternary tetraalkylammonium groups. The alkyl groups are specified as methyl or ethyl groups (Column 4, lines 52-55). Longer alkyl groups are deemed undesirable.

Other solid phase materials for binding nucleic acids based on the anion exchange principle are in present use. These include a silica based material having DEAE head groups (Qiagen) and a silica-NucleoBond AX (Becton Dickinson, Roche-Genopure) based on the chromatographic support described in EP0496822B1. Polymer resins with polymeric-trialkylammonium groups are disclosed in EP 1243649 (GeneScan). Carboxyl-modified polymers for DNA isolation are available from numerous suppliers. Nucleic acids are attracted under high salt conditions and released under low ionic strength conditions.

Materials comprising a solid matrix or substrate such as a filter paper or membrane coated with a composition containing a detergent for causing cellular lysis, a weak base, and a chelating agent are disclosed in U.S. Pat. Nos. 5,496,562, 5,756,126, 6,645,717, and 6,746,841. The coating is applied as a solution and then dried on the matrix. The coating is thus a separate added layer and not an integral part of the material. Additionally, nucleic acid is fixed to the matrix by a subsequent heating step.

Polymeric microcarrier beads having a cationic trimethylamine exterior is described in U.S. Pat. No. 6,214,618. The beads have a relatively large diameter (75-225 μm) and are useful as a support for cell attachment and growth in culture. These beads are not reported to capture or bind nucleic acids.

Magnetically responsive particles have also been developed for use as solid phases in isolating nucleic acids. Several different types of magnetically responsive particles designed for isolation of nucleic acids are known in the art and commercially available from several sources. Magnetic particles which reversibly bind nucleic acid materials directly include MagneSil™ particles (Promega). Magnetic particles are also known that reversibly bind mRNA via covalently attached avidin or streptavidin having an attached oligo dT tail for hybridization with the poly A tail of mRNA.

Various types of magnetically responsive silica-based particles are known for use as solid phases in nucleic acid binding isolation methods. One such type is a magnetically responsive glass bead, preferably of a controlled pore size available as Magnetic Porous Glass (MPG) particles from CPG, Inc. (Lincoln Park, N.J.); or porous magnetic glass particles described in U.S. Pat. Nos. 4,395,271; 4,233,169; 4,297,337; or 6,255, 477. Another type of magnetic useful for binding and isolation of nucleic acids is produced by incorporating magnetic materials into the matrix of polymeric silicon dioxide compounds, e.g. German Patent DE4307262A1; U.S. Pat. Nos. 5,945,525; 6,027,945, and 6,296,937. Magnetic particles comprising iron oxide nanoparticles embedded in a cellulose matrix having quaternary ammonium group is produced commercially by Cortex Biochem (San Leandro, Calif.) as MagaCell-Q™.

Particles or beads having inducible magnetic properties comprise small particles of transition metals such as iron, nickel, copper, cobalt and manganese to form metal oxides which can be caused to have transitory magnetic properties in the presence of magnet. These particles are termed paramagnetic or superparamagnetic. To form paramagnetic or superparamagnetic beads, metal oxides have been coated with polymers which are relatively stable in water. U.S. Pat. No. 4,554,088 discloses paramagnetic particles comprising a metal oxide core surrounded by a coat of polymeric silane. U.S. Pat. No. 5,356,713 discloses a magnetizable microsphere comprised of a core of magnetizable particles surrounded by a shell of a hydrophobic vinylaromatic monomer. U.S. Pat. No. 5,395,688 discloses a polymer core which has been coated with a mixed paramagnetic metal oxide-polymer layer. Another method utilizes a polymer core to adsorb metal oxide such as for example in U.S. Pat. No. 4,774,265. Magnetic particles comprising a polymeric core coated with a paramagnetic metal oxide layer is disclosed in U.S. Pat. No. 5,091,206. The is then further coated with additional polymeric layers to shield the metal oxide layer and to provide a reactive coating. U.S. Pat. No. 5,866,099 discloses the preparation of magnetic particles by co-precipitation of mixtures of two metal salts in the presence of a protein to coordinate the metal salt and entrap the mixed metal oxide. Numerous exemplary pairs of metal salts are described. U.S. Pat. No. 5,411,730 describes a similar process where the precipitated mixed metal oxide is entrapped in dextran, an oligosaccharide.

Alumina (aluminum oxide) particles for irreversible capture of DNA and RNA are disclosed in U.S. Pat. No. 6,291,166. Bound nucleic acid is available for use in solid phase amplification methods such as PCR.

DNA bound to these solid phase materials is eluted in an aqueous solution containing a high concentration of a salt. The nucleic acid solution eluted therefrom must be treated further to remove the salt before it can be used in downstream processes. Nucleic acids bound to silica-based material, in contrast, are freed from the solid phase by eluting with water or a low salt elution buffer. U.S. Pat. No. 5,792,651 describes a composition for chromatographic isolation of nucleic acids which enhances the ability of the nucleic acid in transfection in cells. The composition comprises an aqueous solution containing 2-propanol and optional salts and buffer materials.

Yet other magnetic solid phase materials comprising agarose or cellulose particles containing magnetic micro cores are reported to bind and retain nucleic acids upon treatment with compositions containing high concentrations of salts and polyalkylene glycol (e.g. U.S. Pat. No. 5,898,071 and PCT Publication WO02066993). Nucleic acid is subsequently released by treatment with water or low ionic strength buffer.

Applicants' co-pending U.S. application Ser. Nos. 10/714,763, 10/715,284, 10/891,880, 10/942,491, and 60/638,631 incorporated herein by reference, disclose novel solid phase nucleic acid binding materials, including cleavable materials, and methods of binding and releasing nucleic acids.

Polymeric beads having a tributylphosphonium head group have been described for use as phase transfer catalysts in a three phase system. The beads were prepared from a cross-linked polystyrene. (J. Chem. Soc. Perkin Trans. II, 1827-1830, (1983)). Polymer beads having a pendant trialkylphosphonium group linked to a cross-linked polystyrene resin through alkylene chains and alkylene ether chains have also been described (Tomoi, et al., Makromolekulare Chemie, 187(2), 357-65 (1986); Tomoi, et al., Reactive Polymers, Ion Exchangers, Sorbents, 3(4), 341-9 (1985)). Mixed quaternary ammonium/phosphonium insoluble polymers based on cross-linked polystyrene resins are disclosed as catalysts and biocides (Davidescu, et al., Chem. Bull. Techn. Univ. Timisoara, 40(54), 63-72 (1995); Parvulescu, et al,. Reactive & Functional Polymers, 33(2,3), 329-36 (1997).

### SUMMARY OF THE INVENTION

It is a first object of the present invention to provide simplified, rapid methods for capturing nucleic acids from biological and cellular materials.

It is a further object of the present invention to provide simplified, rapid methods for isolating nucleic acids from biological and cellular materials.

It is a further object of the present invention to provide methods for capturing and isolating nucleic acids from whole blood or blood fractions of an organism.

It is a further object of the present invention to provide methods for capturing and isolating nucleic acids from cell cultures.

It is a further object of the present invention to provide methods for capturing and isolating nucleic acids from biological and cellular materials in under five minutes.

### BRIEF DESCRIPTION OF THE DRAWINGS

FIG. 1 schematically depicts the isolation of nucleic acid from a blood sample according to the present invention.

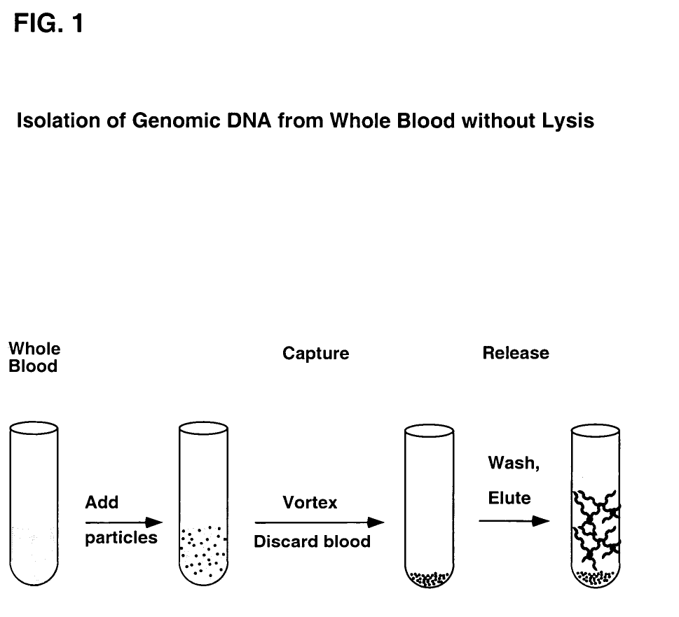

FIG. 2A is an image of a gel showing DNA isolated from human blood samples using the particles of example 2. FIG. 2B is an image of a gel showing amplification of a region of genomic DNA isolated as in FIG. 2A.

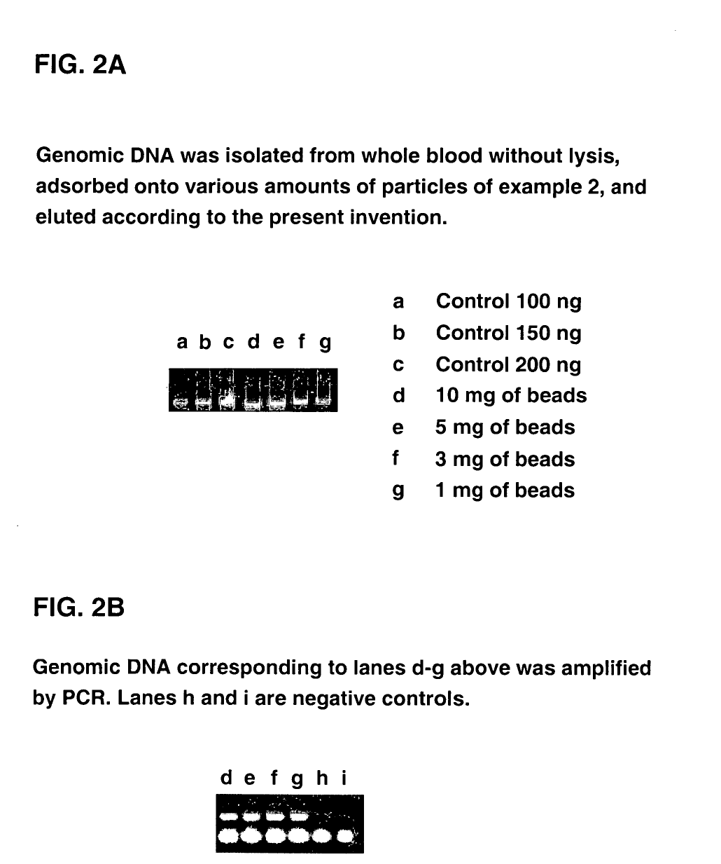

FIG. 3 is an image of a gel showing amplification of a region of genomic DNA isolated according to the present methods using the particles of example 2 or example 7 and various additives.

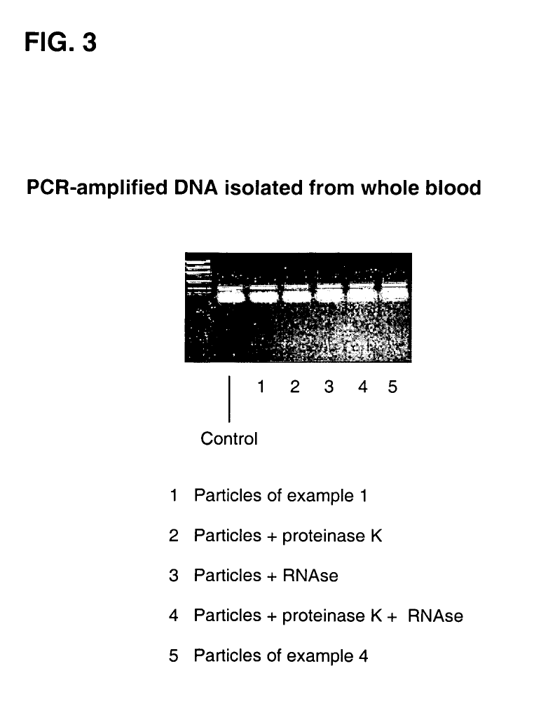

FIG. 4 is an image of a gel showing DNA isolated from human blood samples using various particles of the invention.

FIG. 5A is an image of a gel showing DNA isolated from human blood samples using the particles of examples 2 or 4, eluting with various concentrations of NaOH. FIG. 5B is an image of a gel showing amplification of a region of genomic DNA isolated as shown in 5A.

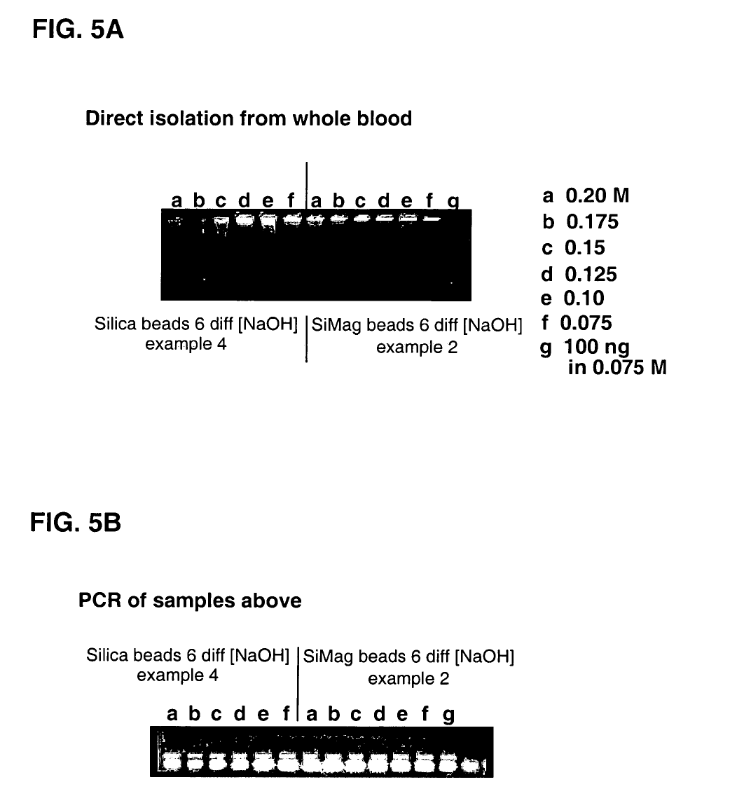

FIG. 6 is an image of a gel showing DNA isolated from human blood samples using various particles of the invention.

### DETAILED DESCRIPTION OF THE INVENTION

Definitions

Alkyl—A branched, straight chain or cyclic hydrocarbon group containing from 1-20 carbons which can be substituted with 1 or more substituents other than H. Lower alkyl as used herein refers to those alkyl groups containing up to 8 carbons.

Aralkyl—An alkyl group substituted with an aryl group.

Aryl—An aromatic ring-containing group containing 1 to 5 carbocyclic aromatic rings, which can be substituted with 1 or more substituents other than H.

Biological material—includes whole blood, anticoagulated whole blood, tissue, cells, cellular content, extracellular nucleic acids, viruses.

Cellular material—intact cells or material, including tissue, containing intact cells of animal, plant or bacterial origin.

Cellular nucleic acid content—refers to nucleic acid found within cellular material and can be genomic DNA and RNA, and other nucleic acids such as that from infectious agents, including viruses and plasmids.

Magnetic—a, micro or bead that is responsive to an external magnetic field. The may itself be magnetic, paramagnetic or superparamagnetic. It may be attracted to an external magnet or applied magnetic field as when using ferromagnetic materials. Particles can have a solid core portion that is magnetically responsive and is surrounded by one or more non-magnetically responsive layers. Alternately the magnetically responsive portion can be a layer around or can be particles disposed within a non-magnetically responsive core.

Oligomer, oligonucleotide—as used herein will refer to a compound containing a phosphodiester internucleotide linkage and a 5′-terminal monophosphate group. The nucleotides can be the normally occurring ribonucleotides A, C, G, and U or deoxyribonucleotides, dA, dC, dG and dT.

Nucleic acid—A polynucleotide can be DNA, RNA or a synthetic DNA analog such as a PNA. Single stranded compounds and double-stranded hybrids of any of these three types of chains are also within the scope of the term.

Release, elute—to remove a substantial portion of a material bound to the surface or pores of a solid phase material by contact with a solution or composition.

Sample—A fluid containing or suspected of containing nucleic acids. Typical samples which can be used in the methods of the invention include bodily fluids such as blood, which can be anticoagulated blood as is commonly found in collected blood specimens, plasma, serum, urine, semen, saliva, cell cultures, tissue extracts and the like. Other types of samples include solvents, seawater, industrial water samples, food samples and environmental samples such as soil or water, plant materials, cells originated from prokaryotes, eukaryotes, bacteria, plasmids and viruses.

Solid phase material—a material having a surface to which can attract nucleic acid molecules. Materials can be in the form of microparticles, fibers, beads, membranes, and other supports such as test tubes and microwells.

Substituted—Refers to the replacement of at least one hydrogen atom on a group by a non-hydrogen group. It should be noted that in references to substituted groups it is intended that multiple points of substitution can be present unless clearly indicated otherwise.

Conventionally, nucleic acids are extracted, isolated and otherwise purified from various sample types by a variety of techniques. Many of these techniques rely on selective adsorption onto a surface of a material with some affinity for nucleic acids. After washing steps to remove other, less strongly bound components, the solid phase is treated with a solution to remove or elute bound nucleic acid(s).

It is frequently necessary to extract and isolate the genomic nucleic acid from a portion of cellular material. Nucleic acids so obtained are used in subsequent processes including amplification, diagnostic tests, analysis of mutations, gene expression profiling and cloning. Samples from which nucleic acids can be isolated by the methods of the present invention include bacterial cultures, bodily fluids, whole blood and blood components, tissue extracts, plant materials, and environmental samples containing cellular materials.

Removal of cellular nucleic acid content by known methods requires the disruption or penetration of cellular membranes or walls in order to access the interior. For this purpose, prior methods employed a cell lysis step. One of these methods uses a reagent for effecting lysis. Lysis solutions are of two types depending on the method of lysis used. One type is an aqueous solution of high pH for alkaline lysis. Another type employs one or more surfactants or detergents to disrupt cell membranes. Lysis solutions can also contain digestive enzymes such as proteinase enzymes to assist in freeing the nucleic acid content of cells. Other methods use a preliminary step of mechanically destroying cells with ultrasound or controlled oscillation with hard particles to disrupt cellular integrity prior to the capture step.

Applicants have developed a new method and new solid phase binding materials which can be used in rapidly capturing and isolating nucleic acids from samples of biological and cellular material, such as viruses, plasmids, extracellular DNA or RNA, whole blood, anticoagulated blood, or bacteria, which do not require any preliminary lysis step. The solid phase binding materials unexpectedly allow the nucleic acid content of cells to be captured in one step. The new methods represent a significant improvement in speed, simplicity, convenience and ease of automation since the use of lysis solutions is eliminated.

In one aspect of the invention there is provided a method of capturing nucleic acids from a sample of biological or cellular material consisting of:
a) providing a solid phase binding material; and
b) combining the solid phase binding material with a sample of biological or cellular material containing nucleic acids for a time sufficient to bind the nucleic acids to the solid phase binding material.

In another aspect of the invention there is provided a method of isolating nucleic acids from a sample of biological or cellular material consisting of:
a) providing a solid phase binding material;
b) combining the solid phase binding material with a sample of cellular material containing nucleic acids for a time sufficient to bind the nucleic acids to the solid phase binding material;
c) separating the sample from the solid phase binding material;
d) optionally washing the solid phase binding material; and
e) releasing the bound nucleic acids from the solid phase binding material.

Unlike prior methods of capturing or isolating nucleic acids from biological samples, no preliminary step of lysing the cells is used. Moreover, no lysis agent, no detergent, surfactant or chaotrope is used or required prior to, concurrent with, or subsequent to contacting the sample with the solid phase binding material. All that is required is to contact the sample of cellular material with the solid phase for a brief period of time. As demonstrated in the examples below, the contact time can be 3 minutes or less and in some cases as little as 30 seconds to capture significant quantities of nucleic acid. The quantities captured can be easily detected after release from the solid phase by common techniques such as gel electrophoresis, fluorescent staining and PCR amplification.

For convenience the solid phase binding material can be added to the sample in water or a solution or buffer known not to cause lysis or cellular degradation. The solid phase binding material can however be added to the sample directly as a dry solid.

In a preferred aspect of the invention there is provided a method of capturing nucleic acids from whole blood of an organism consisting of:
a) providing a solid phase binding material; and
b) combining the solid phase binding material with a sample of whole blood for a time sufficient to bind nucleic acids to the solid phase binding material.

In another preferred aspect of the invention there is provided a method of capturing nucleic acids contained within the leucocytes in whole blood of an organism consisting of:
a) providing a solid phase binding material; and
b) combining the solid phase binding material with a sample of whole blood for a time sufficient to bind nucleic acids from the leucocytes to the solid phase binding material.
   The above methods can be used for isolating the captured nucleic acids by performing the additional steps of:
c) separating the sample from the solid phase binding material;
d) optionally washing the solid phase binding material; and
e) releasing the bound nucleic acids from the solid phase binding material.

Solid phase materials for binding nucleic acids for use with the methods of the present invention comprise a matrix which defines its size, shape, porosity, and mechanical properties, and can be in the form of particles, micro-particles, fibers, beads, membranes, and other supports such as test tubes and microwells. While not wishing to be bound by any particular theory of operation it may be the case that the surface of the solid supports effective in the present methods serve to immobilize nucleic acids directly out of the samples. The term capturing nucleic acid as used herein generally covers whatever mode is in operation to associate the nucleic acids with the solid phase under the conditions of use and contemplates the case where the solid phase binds intact cells as well.

The solid phase material can be any suitable substance having the desired property of binding nucleic acid directly out of samples of cellular material such as whole blood, bacterial cultures. Preferred solid phase materials include silica, glass, sintered glass, controlled pore glass, sintered glass, alumina, zirconia, titania, insoluble synthetic polymers, insoluble polysaccharides, and metallic materials selected from metals, metal oxides, and metal sulfides, as well as magnetically responsive materials coated with silica, glass, synthetic polymers, or insoluble polysaccharides. Exemplary materials include silica based materials coated or functionalized with covalently attached surface functional groups that serve to disrupt cells and attract nucleic acids. Also included are suitably surface-functionalized carbohydrate based materials, and polymeric materials having this surface functionality. Numerous specific materials and their preparation are described in Applicants' co-pending U.S. application Ser. Nos. 10/714,763, 10/715,284, 10/891,880, 10/942,491, and 60/638,631.

In one embodiment the materials further comprise a covalently linked nucleic acid binding portion at or near the surface which permits capture and binding of nucleic acid molecules of varying lengths. By surface is meant not only the external periphery of the solid phase material but also the surface of any accessible porous regions within the solid phase material. Numerous specific materials and their preparation are described in Applicants' co-pending U.S. application Ser. Nos. 10/714,763, 10/715,284, 10/891,880, 10/942,491, and 60/638,631.

In another aspect of the invention there is provided a method of capturing nucleic acids from a sample of biological or cellular material consisting of:
a) providing a solid phase comprising:
   a matrix to which is attached a nucleic acid binding portion;
b) combining the solid phase with a sample of biological or cellular material containing nucleic acids for a time sufficient to bind the nucleic acids to the solid phase.

In another aspect of the invention there is provided a method of isolating nucleic acids from a sample of biological or cellular material consisting of:
a) providing a solid phase comprising:
   a matrix to which is attached a nucleic acid binding portion;
b) combining the solid phase with a sample of biological or cellular material containing nucleic acids for a time sufficient to bind the nucleic acids to the solid phase;
c) separating the sample from the solid phase;
d) optionally washing the solid phase; and
e) releasing the bound nucleic acids from the solid phase.

In another embodiment the materials further comprise a non-covalently associated nucleic acid binding portion at or near the surface which permits capture and binding of nucleic acid molecules of varying lengths. The non-covalently associated nucleic acid binding portion is associated with the solid matrix by electrostatic attraction to an oppositely charged residue on the surface or is associated by hydrophobic attraction with the surface.

The matrix material of these materials carrying covalently or non-covalently attached nucleic acid binding group can be any suitable substance. Preferred matrix materials are selected from silica, glass, insoluble synthetic polymers, insoluble polysaccharides, and metallic materials selected from metals, metal oxides, and metal sulfides as well as magnetically responsive materials coated with silica, glass, synthetic polymers, or insoluble polysaccharides. Exemplary materials include silica based materials coated or functionalized with covalently attached surface functional groups that serve to disrupt cells and attract nucleic acids. Also included are suitably surface-functionalized carbohydrate based materials, and polymeric materials having this surface functionality. Numerous specific materials and their preparation are described in Applicants' co-pending U.S. application Ser. Nos. 10/714,763, 10/715,284, 10/891,880, 10/942,491, and 60/638,631. The surface functional groups serving as nucleic acid binding groups include any groups capable of disrupting cells' structural integrity, and causing attraction of nucleic acid to the solid support. Such groups include, without limitation, hydroxyl, silanol, carboxyl, amino, ammonium, quaternary ammonium and phosphonium salts and ternary sulfonium salt type materials described below. Solid phase materials incorporating amino groups which are protonated at a first lower pH for binding and deprotonated at a second higher pH during release of bound nucleic acid, e.g. materials disclosed in European Patent Specification EP01036082B1, are considered to be within the scope of the solid phase materials useful in the present invention.

For many applications it is preferred that the solid phase material be in the form of particles. Preferably the particles are of a size less than about 50 μm and more preferably less than about 10 μm. Small particles are more readily dispersed in solution and have higher surface/volume ratios. Larger particles and beads can also be useful in methods where gravitational settling or centrifugation are employed. The solid phase preferably can further comprise a magnetically responsive portion which will usually be in the form of magnetic micro-particles that can be attracted and manipulated by a magnetic field. Such magnetic microparticles comprise a magnetic metal oxide or metal sulfide core, which is generally surrounded by an adsorptively or covalently bound layer to which nucleic acid binding groups are covalently bound, thereby coating the surface. The magnetic metal oxide core is preferably iron oxide or iron sulfide, wherein iron is Fe²⁺ or Fe³⁺ or both. Magnetic particles can also be formed as described in U.S. Pat. No. 4,654,267 by precipitating metal particles in the presence of a porous polymer to entrap the magnetically responsive metal particles. Magnetic metal oxides preparable thereby include Fe₃O₄, Fe₂O₃, MnFe₂O₄, NiFe₂O₄, and CoFe₂O₄. Other magnetic particles can also be formed as described in U.S. Pat. No. 5,411,730 by precipitating metal oxide particles in the presence of a the oligosaccharide dextran to entrap the magnetically responsive metal particles. Yet another kind of magnetic particle is disclosed in the aforementioned U.S. Pat. No. 5,091,206. The particle comprises a polymeric core coated with a paramagnetic metal oxide layer and additional polymeric layers to shield the metal oxide layer and to provide a reactive coating. Preparation of magnetite containing chloromethylated Merrifield resin is described in a publication (Tetrahedron Lett.,40 (1999), 8137-8140).

Commercially available magnetic silica or magnetic polymeric particles can be used as the starting materials in preparing magnetic solid phase binding materials useful in the present invention. Suitable types of polymeric particles having surface carboxyl groups are known by the tradenames SeraMag™ (Seradyn) and BioMag™ (Polysciences and Bangs Laboratories). A suitable type of silica magnetic particles is known by the tradename MagneSil™ (Promega). Silica magnetic particles having carboxy or amino groups at the surface are available from Chemicell GmbH (Berlin).

Applicants have prepared magnetically responsive particulate binding materials in accordance with the present invention by linking bare or coated metallic cores with an organic linker group to which is linked a nucleic acid binding (NAB) portion. When using a coated metallic core, a convenient coated core is a silica-coated magnetic core or a glass-coated magnetic core. A preferred magnetically responsive metallic core is provided by magnetite, Fe₃O₄. Magnetite can be acquired commercially or prepared by reaction of iron (II) and iron (III) salts in basic solution according to generally known methods.

Linker groups containing at one terminus a trialkoxysilane group can be attached to the surface of metallic materials or coated metallic materials such as silica or glass-coated magnetic particles. Preferred trialkoxysilane compounds have the formula R¹—Si(OR)₃, wherein R is lower alkyl and R¹ is an organic group selected from straight chains, branched chains and rings and comprises from 1 to 100 atoms. The atoms are preferably selected from C, H, B, N, O, S, Si, P, halogens and alkali metals. Representative R¹ groups are 3-aminopropyl, 2-cyanoethyl and 2-carboxyethyl, as well as groups containing cleavable moieties as described more fully below. In a preferred embodiment, a trialkoxysilane compound comprises a cleavable central portion and a reactive group terminal portion, wherein the reactive group can be converted in one step to a quaternary or ternary onium salt by reaction with a tertiary amine, a tertiary phosphine or an organic sulfide.

It has been found that such linker groups can be installed on the surface of metallic particles and glass or silica-coated metallic particles in a process using fluoride ion. The reaction can be performed in organic solvents including the lower alcohols and aromatic solvents including toluene. Suitable fluoride sources have appreciable solubility in such organic solvents and include cesium fluoride and tetraalkylammonium fluoride salts.

The NAB groups contained in the solid phase binding materials useful in the methods of the present invention appear to serve dual purposes. NAB groups attract and bind nucleic acids, polynucleotides and oligonucleotides of various lengths and base compositions or sequences. They may also serve in some capacity to free nucleic acid from the cellular envelope. Nucleic acid binding groups include, for example, carboxyl, amine and ternary or quaternary onium groups or mixtures of more than one of these groups. Amine groups can be NH₂, alkylamine, and dialkylamine groups. Preferred NAB groups are ternary or quaternary onium groups including quaternary trialkylammonium groups (-QR₃⁺), phosphonium groups (-QR₃⁺) including trialkylphosphonium or triarylphosphonium or mixed alkyl aryl phosphonium groups, and ternary sulfonium groups (-QR₂⁺). The solid phase can contain more than one kind of nucleic acid binding group as described herein. Solid phase materials containing ternary or quaternary onium groups-QR₂⁺ or -QR₃⁺ wherein the R groups are alkyl of at least four carbons are especially effective in binding nucleic acids, but alkyl groups of as little as one carbon are also useful as are aryl groups. Such solid phase materials retain the bound nucleic acid with great tenacity and resist removal or elution of the nucleic acid under most conditions used for elution known in the prior art. Most known elution conditions of both low and high ionic strength are ineffective in removing bound nucleic acids. Unlike conventional anion-exchange resins containing DEAE and PEI groups, the ternary or quaternary onium solid phase materials remain positively charged regardless of the pH of the reaction medium.

Certain preferred embodiments employ solid phase binding materials in which the NAB groups are attached to the matrix through a linkage which can be selectively broken. Breaking the link effectively “disconnects” any bound nucleic acids from the solid phase. The link can be cleaved by any chemical, enzymatic, photochemical or other means that specifically breaks bond(s) in the cleavable linker but does not also destroy the nucleic acids of interest. Such cleavable solid phase materials comprise a solid support portion comprising a matrix selected from silica, glass, insoluble synthetic polymers, insoluble polysaccharides, and metallic materials selected from metals, metal oxides, and metal sulfides. A nucleic acid binding (NAB) portion for attracting and binding nucleic acids is attached to a surface of the solid support by a cleavable linker portion.

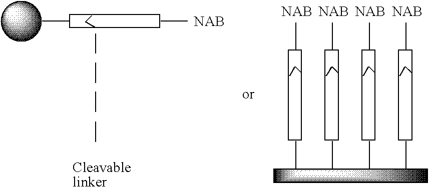

In a preferred embodiment the NAB is a ternary onium group of the formula QR₂⁺X— or a quaternary onium group QR₃⁺X⁻ as described above.

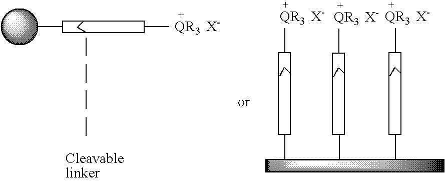

The cleavable linker portion is preferably an organic group selected from straight chains, branched chains and rings and comprises from 1 to 100 atoms. The atoms are preferably selected from C, H, B, N, O, S, Si, P, halogens and alkali metals. An exemplary linker group is a hydrolytically cleavable group which is cleaved by hydrolysis. Carboxylic esters and anhydrides, thioesters, carbonate esters, thiocarbonate esters, urethanes, imides, sulfonamides, and sulfonimides are representative as are sulfonate esters. In a preferred embodiment the cleavable link is treated with an aqueous alkaline solution. Another exemplary class of linker groups are those groups which undergo reductive cleavage such as a disulfide (S—S) bond which is cleaved by thiols such as ethanethiol, mercaptoethanol, and DTT. Another representative group is an organic group containing a peroxide (O—O) bond. Peroxide bonds can be cleaved by thiols, amines and phosphines.

Another representative group is a photochemically cleavable linker group such as nitro-substituted aromatic ethers and esters of the formula 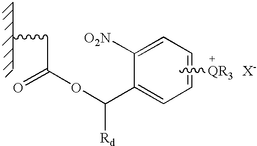
where R_{d} is H, alkyl or phenyl. Ortho-nitrobenzyl esters are cleaved by ultraviolet light according to the well known reaction below.

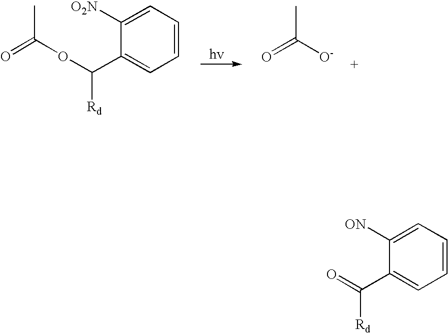

Another representative cleavable group is an enzymatically cleavable linker group. Exemplary groups include esters which are cleaved by esterases and hydrolases, amides and peptides which are cleaved by proteases and peptidases, glycoside groups which are cleaved by glycosidases.

Another representative cleavable group is a cleavable 1,2-dioxetane moiety. Such materials contain a dioxetane moiety which can be decomposed thermally or triggered to fragment by a chemical or enzymatic agent. Removal of a protecting group to generate an oxyanion promotes decomposition of the dioxetane ring. Fragmentation occurs by cleavage of the peroxidic O—O bond as well as the C—C bond according to a well known process. Cleavable dioxetanes are described in numerous patents and publications. Representative examples include U.S. Pat. Nos. 4,952,707, 5,707,559, 5,578,253, 6,036,892, 6,228,653 and 6,461,876. 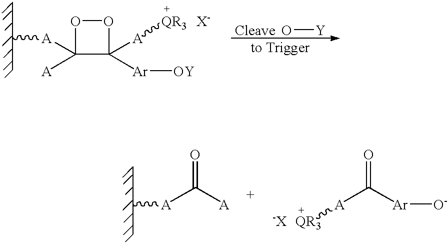
In the alternative, the linked onium group can be attached to the aryl group Ar or to the cleavable group Y. In a further alternative, the linkages to the solid phase and ternary or quaternary onium groups are reversed from the orientation shown.

Another cleavable linker group is an electron-rich C—C double bond which can be converted to an unstable 1,2-dioxetane moiety. At least one of the substituents on the double bond is attached to the double bond by means of an O,S, or N atom. Reaction of electron-rich double bonds with singlet oxygen produces an unstable 1,2-dioxetane ring group which spontaneously fragments at ambient temperatures to generate two carbonyl fragments. Unstable dioxetanes formed from electron-rich double bonds are described in numerous patents and publications exemplified by A. P. Schaap and S. D. Gagnon, J. Am. Chem. Soc., 104, 3504-6 (1982); W. Adam, Chem. Ber., 116, 839-46, (1983); U.S. Pat. No. 5,780,646.

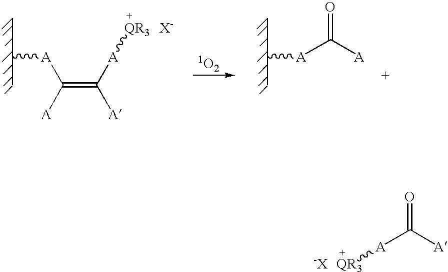

Another group of solid phase materials having a cleavable linker group have as the cleavable moiety a ketene dithioacetal as disclosed in PCT Publication WO 03/053934. Ketene dithioacetals undergo oxidative cleavage by enzymatic oxidation with a peroxidase enzyme and hydrogen peroxide.

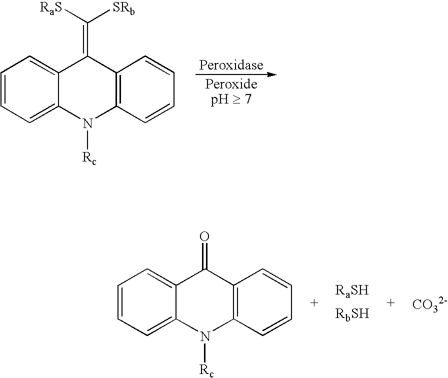

The cleavable moiety has the structure shown, including analogs having substitution on the acridan ring, wherein Rₐ R_{b} and R_{c} are each organic groups containing from 1 to about 50 non-hydrogen atoms selected from C, N, O, S, P, Si and halogen atoms and wherein Rₐ and R_{b} can be joined together to form a ring. Numerous other cleavable groups will be apparent to the skilled artisan.

In another aspect of the invention there is provided a method of capturing nucleic acids from a sample of biological or cellular material consisting of:
a) providing a solid phase comprising:
   a matrix to which is attached, through a selectively cleavable linkage, a nucleic acid binding portion;
b) combining the solid phase with a sample of biological or cellular material containing nucleic acids for a time sufficient to bind the nucleic acids to the solid phase.

There is further provided a method of isolating nucleic acids from a sample of biological or cellular material consisting of:
a) providing a solid phase comprising:
   a matrix to which is attached, through a selectively cleavable linkage, a nucleic acid binding portion;
b) combining the solid phase with a sample of biological or cellular material containing nucleic acids for a time sufficient to bind the nucleic acids to the solid phase;
c) separating the sample from the solid phase; and
d) optionally washing the solid phase; and
e) releasing the bound nucleic acids from the solid phase by selectively cleaving the linker.

In a preferred embodiment the solid phase comprises a matrix selected from silica, glass, insoluble synthetic polymers, and insoluble polysaccharides, and an onium group attached on a surface of the matrix selected from a ternary sulfonium group of the formula QR₂⁺X⁻ where R is selected from C₁-C₂₀ alkyl, aralkyl and aryl groups, a quaternary ammonium group of the formula NR₃⁺X⁻ wherein R is selected from C₁-C₂₀ alkyl, aralkyl and aryl groups, and a quaternary phosphonium group PR₃⁺X— wherein R is selected from C₁-C₂₀ alkyl, aralkyl and aryl groups, and wherein X is an anion,

The step of combining the solid phase with the sample of biological or cellular material containing nucleic acid involve admixing the sample material and the solid phase binding material and, optionally, mechanically agitating the mixture to uniformly distribute the solid phase within the volume of the sample for a time period effective to disrupt the cellular material and bind nucleic acids to the solid phase. It is not necessary that all of the nucleic acid content of the sample become bound to the solid phase, however it is advantageous to bind as much as possible. Agitation of the sample/solid phase mixture can take any convenient form including shaking, use of mechanical oscillators or rockers, vortexing, ultrasonic agitation and the like. The time required to bind nucleic acid in this step is typically on the order of several seconds to a few minutes, but can be verified experimentally by routine experimentation.

The step of separating the sample from the solid phase can be accomplished by filtration, gravitational settling, decantation, magnetic separation, centrifugation, vacuum aspiration, overpressure of air or other gas to force a liquid through a porous membrane or filter mat, for example. Components of the sample other than nucleic acids are removed in this step. To the extent that the removal of other components is not complete, one or more washes can be performed to assist in their complete removal. Wash reagents to remove sample components such as salts, biological or cellular debris, proteins, and hemoglobin include water and aqueous buffer solutions and can contain surfactants.

The step of releasing the bound nucleic acid from the solid phase involves contacting the solid phase material with a solution to release the bound nucleic acids from the solid phase. The solution should dissolve and sufficiently preserve the released nucleic acid. The solution can be a reagent composition comprising an aqueous buffer solution having a pH of about 7-9, optionally containing 0.1-3 M, buffer salt, metal halide or acetate salt and optionally containing an organic co-solvent at 0.1-50% or a surfactant.

The reagent for releasing the nucleic acid from the solid phase after cleavage can alternately be a strongly alkaline aqueous solution. Solutions of alkali metal hydroxides or ammonium hydroxide at a concentration of at least 10⁻⁴ M are effective in cleaving and eluting nucleic acid from the cleaved solid phase. Strongly alkaline solutions are useful in conjunction with solid phase binding materials in which the nucleic acid binding portion is attached to the matrix through a group which can be fragmented or cleaved by covalent bond breakage. Such materials are described below and in the aforementioned co-pending U.S. patent application Ser. Nos. 10/714,763, 10/715,284 and 10/891,880. The release step can be performed at room temperature, but any convenient temperature can be used. Elution temperature does not appear to be critical to the success of the present methods of isolating nucleic acids. Ambient temperature is preferred, but elevated temperatures may increase the rate of elution in some cases.

The methods of solid phase nucleic acid capture can be put to numerous uses. As shown in the particular examples below, both single stranded and double stranded nucleic acid can be captured and released. DNA, RNA, and PNA can be captured and released.

A preferred use of the present methods is in isolation of DNA from whole blood. As described above in the background section, DNA extraction from leucocytes in whole blood, typically is either a cumbersome, multi-step process which is difficult to automate or employs a solid support under solution lysis conditions. The methods of the present invention overcome the limits of prior methods. The method is operationally simple, requiring only the mixing of a blood sample with a solid phase binding material for a brief time to capture the nucleic acid content onto the solid phase material. The entire process can be performed manually in under five minutes. The method is particularly effective and rapid when the solid material is in the form of particles or microparticles. In spite of the simplicity and short times involved, substantial amounts of nucleic acid are captured.

An important advantage of these methods is that they are compatible with many downstream molecular biology processes. Nucleic acid isolated by the present methods can in many cases be used directly in a further process. Amplification reactions such as PCR, Ligation of Multiple Oligomers (LMO) described in U.S. Pat. No. 5,998,175, and LCR can employ such nucleic acid eluents. Isolation of nucleic acid by conventional techniques, especially from bacterial cell culture or from blood samples, involves precipitation by adding a high volume percent of a low molecular weight alcohol. The precipitated materials must then be separated, collected and redissolved in a suitable medium before use. These steps can be obviated by elution of nucleic acid from solid phase binding materials of the present invention using the present methods. It is a preferred practice to use the solution containing the released nucleic acid directly in a nucleic acid amplification reaction whereby the amount of the nucleic acid or a segment thereof is amplified using a polymerase or ligase-mediated reaction.

A wide variety of solid phase binding materials have been described in the foregoing sections and numerous specific exemplary materials are shown in the claimed methods in the following specific examples. The skilled person will be able to determine suitable materials by routine application of the methods described herein.

The present invention further relates to kits containing solid phase binding materials useful in the methods described above. Kits comprise a solid phase binding material and a reagent for releasing nucleic acid from the solid phase. Kits may also include other components such as wash buffers, diluents, or instructions for use.

The solid phase material has the ability to capture nucleic acid directly from biological or cellular material without the use of a lysis solution or coating of lysis agent. Its use to capture nucleic acid does not require any preliminary lysis step and allows the nucleic acid content of biological or cellular material to be captured in one step. In one embodiment the solid phase material is a particulate material or a magnetically responsive particulate material.

### EXAMPLES

Structure drawings when present in the examples below are intended to illustrate only the cleavable linker portion of the solid phase materials. The drawings do not represent a full definition of the solid phase material.

### Example 1

### Synthesis of 4′-Hydroxyphenyl 4-chloromethylthiobenzoate

A 3 L flask was charged with 100.9 g of 4-chloromethylbenzoic acid and 1.2 L of thionyl chloride. The reaction was refluxed for 4 h, after which the thionyl chloride was removed under reduced pressure. Residual thionyl chloride was removed by addition of CH₂Cl₂ and evaporation under reduced pressure.

A 3 L flask containing 113.1 g of 4-chloromethylbenzoic acid chloride was charged with 98.17 g of 4-hydroxythiophenol and 1.5 L of CH₂Cl₂. Argon was purged in and 67.75 mL of pyridine added. After stirring over night, the reaction mixture was diluted with 1 L of CH₂Cl₂ and extracted with a total of 5 L of water. The water layer was back extracted with CH₂Cl₂. The combined CH₂Cl₂ solutions were dried over sodium sulfate and concentrated to a solid. The solid was washed with 500 mL of CH₂Cl₂, filtered and air dried. ¹H NMR (acetone-d₆): δ 4.809 (s, 2H), 6.946-6.968 (d, 2H), 7.323-7.346 (d, 2H), 7.643-7.664 (d, 2H), 8.004-8.025 (d, 2H).

### Example 2

### Synthesis of Magnetic Silica Particles Functionalized with Polymethacrylate Linker and Containing Tributylphosphonium Groups and Cleavable Arylthioester linkage.

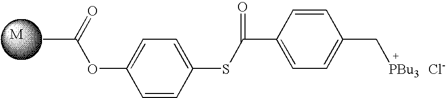

Magnetic carboxylic acid-functionalized silica particles (Chemicell, SIMAG-TCL, 1.0 meq/g, 1.5 g) were placed in 20 mL of thionyl chloride and refluxed for 4 hours. The excess thionyl chloride was removed under reduced pressure. The resin was resuspended in 25 mL of CHCl₃ and the suspension dispersed by ultrasound. The solvent was evaporated and ultrasonic wash treatment repeated. The particles were dried under vacuum for further use.

The acid chloride functionalized particles were suspended in 38 mL of CH₂Cl₂ along with 388 mg of diisopropylethylamine. 4′-Hydroxyphenyl 4-chloromethylthiobenzoate (524 mg) was added and the sealed reaction flask left on the shaker over night. The particles were transferred to a 50 mL plastic tube and washed repeatedly, with magnetic separation, with portions of CH₂Cl₂, CH₃OH, 1:1 CH₂Cl₂/CH₃OH, and then CH₂Cl₂. Wash solutions were monitored by TLC for removal of unreacted soluble starting materials. The solid was air dried before further use.

The resin (1.233 g) was suspended in 20 mL of CH₂Cl₂ under argon. Tributylphosphine (395 mg) was added and the slurry shaken for 7 days. The particles were transferred to a 50 mL plastic tube and washed 4 times with 40 mL of CH₂Cl₂ followed with 4 washes of 40 mL of MeOH and 4 times with 40 mL of CH₂Cl₂. The resin was then air dried yielding 1.17 g of a light brown solid.

### Example 3

### Synthesis of Silicate Linker Functionalized with a Cleavable Linker Containing Tributylphosphonium Groups

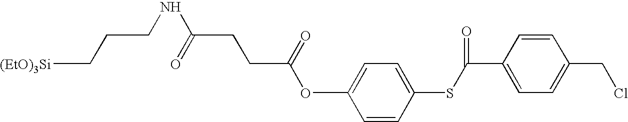

A solution of 3-aminopropyltriethoxysilane (13.2 mL) in 75 mL of heptane and 13 mL of ethanol was placed under Ar and stirred with 5.5 g of succinic anhydride. The reaction was refluxed for 4.5 h and then cooled to room temperature over night. The solvent was removed yielding the amide product as a clear oil.

A solution of EDC hydrochloride (4.0 g) and 2.86 g of the product above in 100 mL of CH₂Cl₂ was placed under Ar and stirred for 1 h before adding 5.5 g of 4′-hydroxyphenyl 4-chloromethylthiobenzoate (example 1). The reaction was stirred over night. The reaction mixture was chromatographed onto 150 g of silica, eluted with 1-2% EtOH/CH₂Cl₂ yielding 1.84 g of the coupled product as a white solid.

### Example 4

### Synthesis of Silica Particles Functionalized with a Cleavable Linker Containing Tributylphosphonium Groups

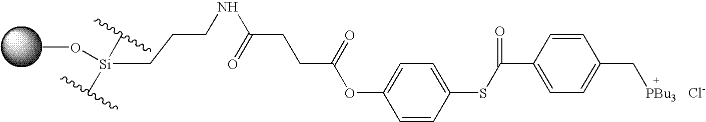

The product of example 3 (1.84 g) in 50 mL of dry toluene was added via cannula to a flask containing 3.83 g of oven-dried silica under a blanket of Ar. The reaction was refluxed over night. After cooling to room temperature, the silica was filtered off, washed with 500 mL of CH₂Cl₂₁ and vacuum dried for 4 h.

The derivatized silica having chlorobenzyl end groups (2.0 g) in 50 mL of CH₂Cl₂ was mixed with 8.0 g of tributylphosphine. The reaction mix was stirred under Ar for 2 d. The silica was filtered off, washed with CH₂Cl₂ and hexanes, and vacuum dried for several hours.

### Example 5

### Synthesis of a Magnetic Particles Coated with a Cleavable Linker Containing Tributylphosphonium Groups

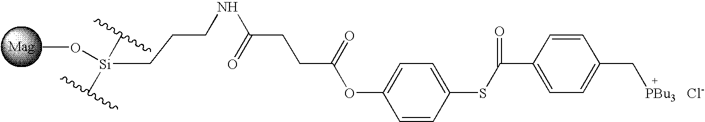

The silicate linker of example 3 (0.25 g) was reacted with 0.5 g of Fe₃O₄ particles by stirring in refluxing toluene under Ar over night. After cooling, the solids and toluene solution were transferred to a 50 mL centrifuge tube. Solids were attracted to an external magnet, the toluene decanted, and the solids washed 3× with toluene and 3× with CH₂Cl₂.

The particles of the previous step (0.40 g) were suspended in 25 mL of CH₂Cl₂. Tributylphosphine (1.6 g) was added to the suspension and the vessel sealed before placing on an orbital shaker for 1.5 days. The solid was subjected to the “magnetic wash” described above, yielding a black powder.

### Example 6

### Synthesis of a Magnetic Silica Particles Coated with a Cleavable Linker Containing Tributylphosphonium Groups

A nucleic acid binding material was prepared by passively adsorbing a cleavable nucleic acid binding group onto the surface of silica s.

Stearic acid (1.33 g) was refluxed in 10 mL of SOCl₂ for 2 h. The excess SOCl₂ was removed under reduced pressure producing stearoyl chloride as a brown liquid.

Stearoyl chloride was dissolved in 10 mL of CH₂Cl₂ and added to a solution of 1.0 g of 4′-hydroxyphenyl 4-chloromethylthiobenzoate, prepared as described in Example 1, and 1.56 mL of diisopropylethylamine in 30 mL of CH₂Cl₂ and the mixture stirred over night. The solvent was removed and residue subject to column chromatography using 1:1 hexane/CH₂Cl₂ as eluent. The stearoyl ester (1.43 g) was isolated as a white solid.

A solution of the above product (1.43 g) and tributylphosphine (1.27 mL) in 30 mL of CH₂Cl₂ was stirred under an Ar atmosphere for 2 d. After removal of CH₂Cl₂ the residue was washed with 6×50 mL of ether, redissolved in CH₂Cl₂ and precipitated with ether producing 1.69 g of the phosphonium salt product. This material was found to be insoluble in water.

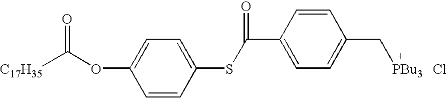

The phosphonium salt (0.6 g) was dissolved in 6 mL of CH₂Cl₂ and added to 6.0 g of silica gel with agitation. Evaporation of solvent produced the nucleic acid binding material.

### Example 7

### Synthesis of Magnetic Particles Having a Polymeric Layer Containing Polyvinylbenzyl Tributylphosphonium Groups

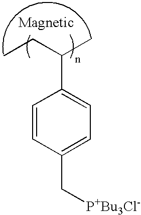

Magnetic Merrifield peptide resin (Chemicell, SiMag Chloromethyl, 100 mg) was added to 2 mL of CH₂Cl₂ in a glass vial. Tributylphosphine (80 μL) was added and the slurry was shaken at room temperature for 3 days. A magnet was placed under the vial and the supernatant was removed with a pipet. The solids were washed four times with 2 mL of CH₂Cl₂ (the washes were also removed by the magnet/pipet procedure). The resin was air dried (93 mg).

### Example 8

### Synthesis of Polymethacrylate Polymer Particles Containing Tributylphosphonium Groups and Cleavable Arylthioester Linkage

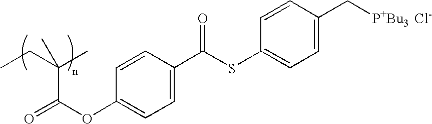

Poly(methacryloyl chloride) particles (1.0 meq/g, 1.5 g) were placed in 75 mL of CH₂Cl₂ containing 2.45 g of diisopropylethylamine. Triethylamine (1.2 g) was added. 4′-Hydroxyphenyl 4-chloromethylthiobenzoate (4.5 g) was added and the sealed reaction mixture was stirred overnight at room temperature. The slurry was filtered and the resin washed with 10 mL of CH₂Cl₂, 200 mL of acetone, 200 mL of MeOH, 2×100 mL of 1:1 THF/CH₂Cl₂, 250 mL of THF, 250 mL of CH₂Cl₂, 250 mL of hexane. The resin was air dried for further use.

The resin (1.525 g) was suspended in 25 mL of CH₂Cl₂ under argon. Tributylphosphine (1.7 g) was added and the slurry stirred for 4 days. The resin was filtered and washed 4 times with 225 mL of CH₂Cl₂ followed by 175 mL of hexane. The resin was then air dried yielding 1.68 g of solid.

### Example 9

### Synthesis of a Polystyrene Polymer Containing Tributylphosphonium Groups

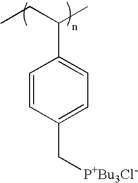

Merrifield peptide resin (Sigma, 1.1 meq/g, 20.0 g) which is a crosslinked chloromethylated polystyrene was stirred in 200 mL of CH₂Cl₂/DMF (50/50) under an argon pad. An excess of tributylphosphine (48.1 g, 10 equivalents) was added and the slurry was stirred at room temperature for 7 days. The slurry was filtered and the resulting solids were washed twice with 200 mL of CH₂Cl₂. The resin was dried under vacuum (21.5 g). Elemental Analysis: Found P 2.52%, Cl 3.08%; Expected P 2.79%, Cl 3.19%: P/Cl ratio is 0.94.

### Example 10

### Synthesis of a Polystyrene Polymer Containing Tributylammonium Groups

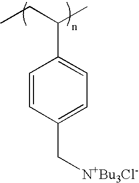

Merrifield peptide resin (Aldrich, 1.43 meq/g, 25.1 g) was stirred in 150 mL of CH₂Cl₂ under an argon pad. An excess of tributyl amine (25.6 g, 4 equivalents) was added and the slurry was stirred at room temperature for 8 days. The slurry was filtered and the resulting solids were washed twice with 250 mL of CH₂Cl₂. The resin was dried under vacuum (28.9 g). Elemental Analysis: Found N 1.18%, Cl 3.40%; Expected N 1.58%, Cl 4.01%: N/Cl ratio is 0.88.

### Example 11

### Synthesis of Silica Particles Functionalized With Tributylphosphonium Groups

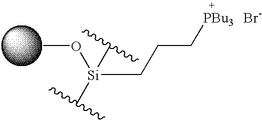

Silica gel dried for 1 h at 110° C. under Ar (4.82 g) was added to 50 mL of CH₂Cl₂ along with 2.79 g of Et₃N. The mixture was stirred for 20 min after which 2.56 g of 3-bromopropyltrichlorosilane was added, causing an exotherm. The mixture was stirred for 24 h, filtered and the solid washed sequentially with 3×40 mL of CH₂Cl₂, 4×40 mL of MeOH and 2×40 mL of CH₂Cl₂. The solid was air-dried over night and weighed 6.13 g.

The functionalized silica prepared above (5.8 g) in 50 mL of CH₂Cl₂ was stirred with 5.33 mL of tributylphosphine for 10 days. The mixture filtered and the solid washed with 7×50 mL of acetone. Air drying the solid produced 5.88 g of the product.

### Example 12

### Controlled Cleavage of Linker in Nab Material of Example 6

The coated silica material of example 6 (70 mg) was suspended in 1.0 mL of D₂O and mixed by vortexing for 3 min. Analysis of the water solution by ¹H NMR showed no release of material into solution.

Treatment of the silica suspension with 40 μL of 40% NaOD and vortexing for 3 min and NMR analysis of the supernatant showed cleavage of the linker and release from the silica into solution.

### Example 13

### Synthesis of Siloxane-Coated Magnetite

Magnetite 1.00 g (Alfa Aesar) in 100 mL of anhydrous ethanol was reacted with 3.2 mL of TEOS, 3.32 g of CsF and 1.0 mL of water under Ar at reflux for two hours. The cooled reaction mixture was decanted and the solids washed magnetically 4× with ethanol and 5× with CH₂Cl₂. Drying the solids under Ar yielded 3.14 g of solid. A 1.0 g portion of this material was washed sequentially with 5×50 mL of deionized water and 5×50 mL of methanol. Drying produced 0.67 g of solid.

### Example 14

### Synthesis of Magnetic Particles Containing Polyvinylbenzyl Tributylphosphonium Groups

Iron oxide, 1.0 g, was dispersed in 100 mL of ethanol by the aid of an ultrasonic bath. The reaction vessel was charged with 1.50 mL of TEOS, 1.65 g of p-(chloromethyl)phenyltrimethoxysilane (Gelest), 3.32 g of cesium fluoride and 1.0 mL of deionized water. The reaction mixture was stirred at reflux under an Ar atmosphere for two hours. The mixture was cooled to room temperature, the solvent decanted and the solid washed magnetically five times with ethanol and five times with CH₂Cl₂. Drying the solid with a stream of Ar yielded 2.96 g of product.

The particles of the previous step (0.50 g) were suspended in 20 mL of CH₂Cl₂. Tributylphosphine (0.5 mL) was added to the suspension and the vessel sealed before placing on an orbital shaker over night. The solid was washed magnetically five times with CH₂Cl₂. Drying the solid with a stream of Ar yielded 0.48 g of product.

### Example 15

### Synthesis of Functionalized Siloxane-Coated Magnetite

Magnetite 1.00 g (Alfa Aesar) in 200 mL of anhydrous ethanol was reacted with 3.0 mL of 3-(triethoxysilyl)-propionitrile, 3.32 g of CsF and 1.0 mL of water under Ar at reflux for two hours. The cooled reaction mixture was decanted and the solids washed magnetically 4× with ethanol and 5× with CH₂Cl₂. Drying the solids under Ar yielded 2.46 g of solid.

### Example 16

### Synthesis of Functionalized Siloxane-Coated Controlled Pore Glass

The silicate linker of example 2 (1.84 g) in 50 mL of dry toluene was added via cannula to a flask containing 3.83 g of oven-dried controlled pore glass Prime Synthesis native CPG (0.5 g) under a blanket of Ar. The reaction was refluxed over night. After cooling to room temperature, the glass was filtered off, washed with 500 mL of CH₂Cl₂, and vacuum dried for 4 h.

The derivatized glass having chlorobenzyl end groups (2.0 g) in 50 mL of CH₂Cl₂ was mixed with 8.0 g of tributylphosphine. The reaction mix was stirred under Ar for 2 d. The glass was filtered off, washed with CH₂Cl₂ and hexanes, and vacuum dried for several hours.

### Example 17A

### Synthesis of Functionalized Magnetic Polystyrene

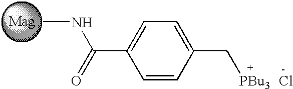

A suspension of amine-functionalized magnetic polystyrene beads (Spherotech) in H₂O (4×1 mL containing 25 mg each) was taken and added to two 1.5 mL tubes. The supernatant was removed and the beads were washed with 3×1 mL of 0.1 M MES buffer, pH 4.0. To each tube was added 600 μL of MES buffer and 28 mg EDC (0.147 mmol) and a solution of 50 mg of 4-chloromethylbenzoic acid in 300 μL of DMF (0.174 mmol). After 1 day of stirring, the tubes were sonicated for 1 h and kept on a magnetic rack. The reaction mixture was transferred to two 50 mL tubes and diluted to 40 mL with H₂O. The beads were washed magnetically with water (4×40 mL), 1:1 CH₃OH:H₂O (40 mL), and CH₃OH (3×40 mL) and were allowed to dry in the tubes.

The above solid (90 mg) was placed in a 1.5 mL tube and 800 μL of CH₃OH was added. A solution of 30 mg PBu₃ in 200 μL of CH₃OH was added to this suspension. The reaction mixture was sonicated 30 min and stirred at room temperature. After 9 days of stirring, the supernatant was removed by keeping on a magnet. Beads were washed magnetically with water (4×1 mL), CH₃OH (4×1 mL), and water (1×1 mL). Then 1 mL of water was added to the beads to make a 10 mg/mL stock suspension.

### Example 17B

### Alternate Synthesis of Functionalized Magnetic Polystyrene

A solution of 1.00 g of 4-chloromethylbenzoic acid (5.86 mmol) and 3,00 mL of tributylphosphine (12.0 mmol) in 30 mL of acetone was stirred under Ar over night causing formation of a white precipitate identified as 4-carboxybenzyltributylphosphonium chloride by ¹H NMR. the solid was collected by filtration and washed with acetone and then with hexanes. Yield 2.19 g, 89%.

A suspension of amine-functionalized magnetic polystyrene beads (Spherotech) in H₂O (2×1 mL containing 25 mg each) was taken and added to two 1.5 mL tubes. The supernatant was removed and the beads were washed with 3×1 mL of 0.1 M MES buffer, pH 4.0. To each tube was added 600 μL of MES buffer and a solution of 30 mg of 4-carboxybenzyltributylphosphonium chloride (80 μmol) in 200 μL of DMF/200 μL of MES buffer and EDC (15 mg, 78 μmol). The tubes were sonicated for 30 min and placed on a shaker for 1 day. The supernatant was removed by keeping on a magnet. Beads were washed magnetically with water (4×1 mL), CH₃OH (4×1 mL), and water (1 mL) and then water was added to make a 100 mg/mL stock suspension.

### Example 18

### Synthesis of Functionalized Magnetic Polystyrene

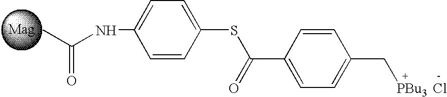

A solution of 1.0 g of 4′-aminophenyl 4-chloromethylbenzenethiocarboxylate (prepared by EDC coupling of 4-chloromethylbenzoic acid and 4-aminothiophenol) in 30 mL of acetone and 2.0 g of tributylphosphine was stirred under Ar over night. The precipitate which had formed was filtered of and washed with acetone and hexanes. Yield of the phosphonium salt was 1.41 g, 82%.

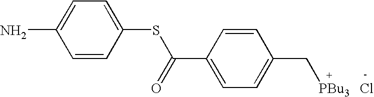

Magnetic carboxylated polystrene resin, 1 mL from a 100 mg/mL suspension, was decanted and the solid washed with 3×1 mL 0.1 M MES buffer, pH 4.0. The product of the previous step (50 mg) was dissolved in 400 μL of DMF and 400 μL of MES buffer. This solution was added to a suspension of the beads in 200 μL of MES buffer. Then 28 mg EDC was added and the suspension was sonicated for 30 min and placed on a shaker. After one day of stirring, the reaction mixture was removed. Beads were washed magnetically with 4×1 mL H₂O, 4×1 mL CH₃OH, 1×1 mL H₂O. The beads were suspended in H₂O (100 mg/mL).

### Example 19

### Synthesis of Functionalized Magnetic Polystyrene

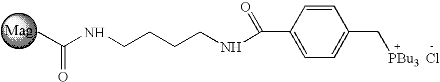

The supernatant was removed from 1 mL of a 100 mg/mL suspension of magnetic carboxylated polystrene resin and the solids were washed with 3×1 mL of 0.1 M MES buffer, pH 4.0. The beads were suspended in 800 μL MES and a solution of 63 mg of 1,4-diaminobutane in 200 μL of MES buffer was added. EDC (28 mg, 0.147 mmol) was added and beads were sonicated for 30 min and then stirred at room temperature. The reaction mixture was separated from beads magnetically. The beads were then washed magnetically with 4×1 mL water and 4×1 mL of MES buffer.

50 mg of 4-carboxybenzyltributylphosphonium chloride (0.134 mmol) was dissolved in a 1:1 mixture of 400 μL DMF/MES buffer and added to a suspension of the above beads in 600 μL of MES buffer. Tube was sonicated for 30 min and kept on a shaker. After one day of stirring, the solution was decanted and the beads were washed magnetically with water (4×1 mL), CH₃OH (4×1 mL), and water (1 mL) and water was added to make a 100 mg/mL stock suspension.

### Example 20

### Synthesis of Magnetic Polymer with Electrostatically Associated Phosphonium Group

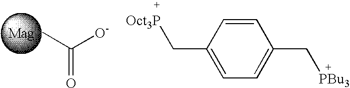

The supernatant was removed from 1 mL of a 100 mg/mL suspension of magnetic carboxylated polystrene resin. The beads were agitated with 1 mL of 0.1M NaOH for 5 min. After decanting the solution the beads were washed with 1 mL of water. A solution of 20 mg of Plus Enhancers (prepared as described in U.S. Pat. No. 5,451,437) in 400 μL of water was added to the beads and the mixture was shaken for 5 min. After removing the supernatant, the beads were washed with 3×1 mL of water and water was added to make a 100 mg/mL stock suspension.

### Example 21

### Synthesis of Functionalized Magnetic Polymer

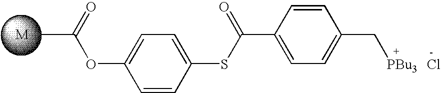

An aliquot of beads (Dynal magnetic COOH beads, Cat. No. G03810)) containing 25 mg of solid was decanted by the aid of a magnet. Beads were then washed with 3×1 mL of water, and 3×1 mL CH₃CN before drying overnight. The beads were suspended in 800 μL of CH₂Cl₂ to which was added 15 mg of EDC (78 μmol). A solution of the compound of Example 1 (30 mg) in 200 μL of DMF was added to the mixture. The tube was sonicated for 30 min and shaken over night. The supernatant was removed and the beads were washed magnetically with 4×1 mL of CH₂Cl₂₁ 1 mL of 1:1 MeOH:CH₂Cl₂, 3×1 mL of MeOH and 4×1 mL of CH₂Cl₂. The beads were dried in air overnight.

The beads were suspended in 1 mL of CH₂Cl₂ to which was added 30 μL of tributylphosphine. The reaction mixture was sonicated for 30 min and shaken for a total of 5 days. The solvent was decanted by keeping on a magnet. Beads were washed magnetically with 4×1 mL of CH₂Cl₂, 3×1 mL of CH₃OH, and 2×1 mL of water. A stock solution of beads (25 mg/mL) was made by adding 1 mL of water.

### Example 22

### Synthesis of Functionalized Magnetic Polymer

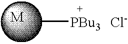

An aliquot of Dynal tosyl activated beads (1 mL of a 97.5 mg/mL stock, Cat. No. F68710) was placed in a 1.5 mL tube and the solvent was removed using a magnetic rack. The beads were washed with 2×1 mL of water and 5×1 mL of CH₃OH. Tributylphosphine (100 μL) was added to the beads in a suspension of 1 mL of CH₃OH. The tube was placed on a shaker at room temperature. After 9 days the supernatant was removed by aid of a magnet. The beads were washed with 4×1 mL of water, 4×1 mL of CH₃OH, and 1 mL of water. Then 1 mL of water was added to the beads to prepare a 100 mg/mL stock solution.

### Example 23

### Synthesis of Magnetic Polymer Particles with a Cleavable Linker Containing Tributylphosphonium Groups Non-Covalently Bound to the Particle

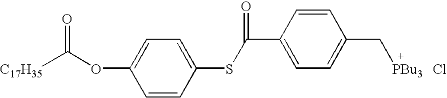

From a stock solution of (100 mg/mL) magnetic carboxylated polystrene particles, 500 μL was placed in a 1.5 mL tube on a rack and the supernatant was removed. The beads were washed with 3×500 μL of water and 4×500 μL of MeOH. The compound shown above, 10 mg, was dissolved in 100 μL of CH₃OH, added to the beads and the solvent allowed to evaporate in air.

### Example 24

### Synthesis of Magnetic Silica Particles Functionalized with Polymethacrylate Linker and Containing tris(carboxyethyl)phosphonium Groups and Cleavable Arylthioester Linkage

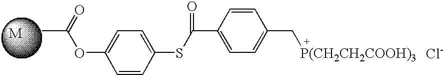

Magnetic carboxylic acid-functionalized silica particles (Chemicell, SiMAG-TCL, 1.0 meq/g, 1.5 g) were functionalized as described in example 2 excluding the last step. This material (116.5 mg) was suspended in 10 mL of CH₂Cl₂ by sonication for 3 min. Tris(2-carboxyethyl)-phosphine (66.8 mg) and 32 μL of triethylamine were added and the slurry shaken for 7 days. The particles were transferred to a flask and washed 3 times with 20 mL of CH₂Cl₂ followed with 4 washes of 20 mL of MeOH and 2 times with 20 mL of CH₂Cl₂. The solid was then air dried yielding 109 mg of material.

### Example 25

### Synthesis of Functionalized Magnetic Polymethacrylate Particles

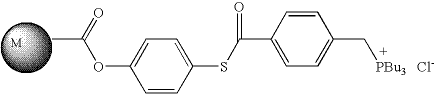

Magnetic particles from 40 mL of Sera-Mag Magnetic Carboxylate-Modified microparticle suspension (Seradyn) were magnetically collected and the supernatant decanted. The particles were magnetically washed with 4×50 mL of type I water and then with 4×50 mL of acetonitrile. After the final wash the particles were dried yielding 1.93 grams of brown solid.

A 100 mL round bottom flask was charged with 1.02 g of the particles, 0.2899 grams (1.5 mmol) of EDC, 0.5058 grams (1.8 mmol) of the linker of example 1, and 50 ml of CH₂Cl₂. The mixture was sonicated for 10 min and placed on an orbital shaker to stir (170 rpm) for 11 days with periodic sonication for 5 min to ensure homogeneity. The product was collected magnetically and the solid was magnetically washed with 4×50 mL of CH₂Cl₂, 50 mL of 1:1 CH₂Cl₂/MeOH, 4×50 mL of MeOH, 50 mL of 1:1 CH₂Cl₂/MeOH, and 4×50 mL of CH₂Cl₂. The solid was dried yielding 0.951 g of brown solid.

A 50 ml round bottom flask was charged with 0.8993 g of the above material and 20 mL of CH₂Cl₂. The mixture was sonicated for five min and 0.24 g (1.2 mmol) of tributylphosphine added. The mixture was sonicated for another 15 min after this addition and stirred on an orbital shaker for 7 days with periodic sonication. The product was then collected magnetically and washed 4×50 mL of CH₂Cl₂, 50 mL of 1:1 CH₂Cl₂/MeOH, 4×50 mL of MeOH, 50 mL of 1:1 CH₂Cl₂/MeOH, and 4×50 mL of CH₂Cl₂. The solid was dried yielding 0.8801 grams of brown solid.

### Example 26

### Synthesis of Magnetic Functionalized Polymer by Inclusion of Iron Oxide in Preformed Polymer

A mixture of 1.00 g of the polymer product of example 9 and 0.2 g of iron oxide were mixed to homegeneity before adding 20 mL of CH₂Cl₂. The mixture was sonicated for 15 min, diluted to 100 mL with hexanes and filtered. The collected solids were washed with 200 mL of acetone and 400 mL of water until no color came off and then with 200 mL of acetone. The solid was magnetically washed with 4×40 mL of acetone. The solid was collected by filtration, washed with acetone and dried. There was 0.7 g of solid which when examined under a microscope showed only a very small amount of free magnetite.

### Example 27

### Preparation of Functionalized Controlled Pore Glass

0.5 g native controlled pore glass (Prime Synthesis, Aston, PA 18-50 mesh, 500 Å pore size) was combined with the triethoxysilane linker of example 3 (0.25 g, 0.43 mmol) and 50 mL anhydrous toluene. The mixture was refluxed for 18 h under a blanket of Ar. After cooling to room temperature, the glass particles were isolated by suction filtration, and washed with 0.2 L toluene and 0.2 L CH₂Cl₂. After air-drying overnight, 0.52 g of glass particles was obtained.

A 0.450 g portion of the above glass particles was combined with 10 mL CH₂Cl₂ and PBu₃ (0.91 g, 4.5 mmol). The mixture was placed on a rotary orbital shaker at room temperature and shaken for 3 d. The glass particles were isolated by suction filtration and washed successively with 0.2 L CH₂Cl₂, 0.2 L MeOH, and 0.3 L CH₂Cl₂. After air-drying overnight, 0.454 g of glass particles was obtained.

Similar procedures were followed for CPG having a size of 120-200 mesh and a pore size of either 500 or 1000 Å.

### Example 28

### Preparation of Functionalized Sintered Glass

Four small sintered glass filters (ca. 35 mg ea, R & H Filter Co.) were pre-treated in succession with 20% aqueous NaOH, 1 N HCl, water, and MeOH. After drying, the frits were combined with the triethoxysilane of example 3 (0.32 g. 0.55 mmol), 10 mL toluene, and 10 μL H₂O. The mixture was refluxed for 16 h under a blanket of Ar. After cooling to room temperature, the frits were removed and washed successively with CH₂Cl₂₁ MeOH, and CH₂Cl₂.

The above glass filters were combined with 10 mL CH₂Cl₂ and PBu₃ (0.20 g, 0.99 mmol). The mixture was placed on a rotary orbital shaker at room temperature and shaken for 7 d. The filters were removed and washed successively with CH₂Cl₂₁ MeOH, and CH₂Cl₂.

### Example 29

### Preparation of Acridinium Amide Functionalized Silica Gel

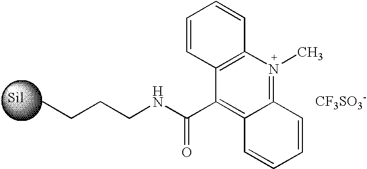

3-Aminopropyl silica gel was either obtained commercially (Silicycle, Quebec, Canada) or prepared by refluxing “silica gel 60” with excess 3-aminopropyl triethoxysilane in toluene overnight. The 3-aminopropyl derivatized silica gel (1.00 g, loading ca. 1 mmol/g) was suspended in CH₂Cl₂ (15 mL) under an Ar blanket. N,N-diisopropylethylamine (1.5 mL, 8.61 mmol) was added via syringe, followed by acridine 9-acid chloride (360 mg, 1.49 mmol). The mixture was placed on a rotary orbital shaker and shaken at room temperature for 2 h. The dark brown reaction product was suction filtered on a sintered glass funnel and washed sequentially with CH₂Cl₂ (0.2 L), 20% (v/v) MeOH:CH₂Cl₂ (0.2 L), and CH₂Cl₂ (0.25 L). After air-drying, 1.16 g of a powdery solid was obtained.

The material prepared above (1.00 g) was suspended in CH₂Cl₂ (15 mL) and swirled to disperse the solid. Methyl triflate (0.17 mL, 1.5 mmol) was added and the reaction was sealed with a rubber septum. The mixture was placed on a rotary orbital shaker and shaken at room temperature for 16 h. The resulting mixture was suction filtered on a sintered glass funnel and washed sequentially with CH₂Cl₂ (0.2 L), MeOH (0.2 L), and CH₂Cl₂ (0.25 L). After air-drying, 1.02 g of a powdery solid was obtained.

### Example 30

### Preparation of Functionalized Silica Gel

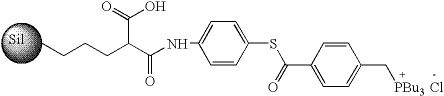

A solution of 2-(3-triethyoxysilylpropyl)succinic anhydride, 2.00 g and 4′-aminophenyl 4-chloromethylbenzenethiocarboxylate, 1.82 g in 30 mL of CH₂Cl₂ was stirred over night at room temperature. The solvent was evaporated leaving 3.8 g of a waxy solid. This solid was mixed with 1.0 g of silica in 170 mL of toluene and heated qt 70° C. with stirring for 16 h. After cooling, the yellow solid was filtered and washed sequentially with acetone (5×50 mL), CH₂Cl₂ (5×50 mL), MeOH (5×50 mL), and CH₂Cl₂ (2×50 mL). After air-drying, 3.02 g of a yellowish solid was obtained.

A suspension of the above material, 2.00 g in 100 mL of CH₂Cl₂ was sonicated for 5 min, put under a blanket of argon and treated with 1.40 mL of tributylphosphine. This mixture was stirred for 7 days, filtered and washed sequentially with CH₂Cl₂ (4×50 mL), MeOH (4×50 mL), and CH₂Cl₂ (4×50 mL). After air-drying, 2.04 g of a yellowish solid was obtained.

### Example 31

### Capture of DNA from Whole Human Blood

A 10 mg portion of the particles was mixed with 70 μL of whole human blood in a tube. The tube was vortexed for 15 s, held for 5 min at room temperature, and again vortexed for 15 s. The mixture was diluted with 300 μL of 10 mM tris buffer, pH 8.0 and the liquid removed from the particles, with the aid of a magnet when magnetically responsive particles were employed. Magnetic separations were performed with a Dynal MPC-5 magnetic rack.

### Example 32

### Isolation of DNA from Whole Human Blood

Nucleic acid captured on the solid phase binding material according to the procedure of the preceding example was washed three times with 500 μL of 10 mM tris buffer, pH 8.0, discarding the supernatant each time. Nucleic acids were removed from the particles by eluting with 100 μL of 0.1 M NaOH at 37° C. for 5 min. Other concentrations of NaOH were also effective.

### Example 33

### Rapid Isolation Protocol

A 1 mg portion of the particles was mixed with 100 μL of whole human blood in a tube. The tube was vortexed for 30 s. The mixture was diluted with 300 μL of 10 mM tris buffer, pH 8.0 and the liquid removed from the particles, with the aid of a magnet when magnetically responsive particles were employed. Nucleic acid captured on the solid phase binding material according to the procedure of the preceding example was washed three times with 500 μL of 10 mM tris buffer, pH 8.0, discarding the supernatant each time. Nucleic acids were removed from the particles by eluting with 50 μL of 0.05 M NaOH at room temperature for 30 s.

### Example 34

### Fluorescent Assay Protocol

Supernatants and eluents were analyzed for DNA content by a fluorescent assay using PicoGreen to stain DNA. Briefly, 10 μL aliquots of solutions containing or suspected to contain DNA are incubated with 190 μL of a fluorescent DNA “staining” solution. The fluorescent stain was PicoGreen (Molecular Probes) diluted 1:400 in 0.1 M tris, pH 7.5, 1 mM EDTA. Fluorescence was measured in a microplate fluorometer (Fluoroskan, Labsystems) after incubating samples for at least 5 min. The filter set was 480 nm and 535 nm. Positive controls containing a known amount of the same DNA and negative controls were run concurrently. For experiments where nucleic acid was eluted in 100 μL of 0.1 M NaOH, a 10 μL aliquot was used. For experiments where nucleic acid was eluted in 50 μL of 0.05 M NaOH, a 5 μL aliquot was used.

### Example 35

### Gel Electrophoresis Protocol

Either 0.75% or 1.5% agarose gels were prepared for analysis of nucleic acid eluents. The appropriate amount of agarose was dissolved in 10 mL of TAE buffer by boiling for 2 min. Upon cooling to 50-60° C. a solution (20 μL) of 1 mg/mL ethidium bromide was added and the gel was poured. Each sample (ca. 12 μL) was mixed with 2 μL of 6× loading buffer containing 0.25% bromophenol blue, 0.25% xylene cyanol and 30% glycerol. Gels were run at 70 V.

### Example 36

### PCR Amplification of Genomic DNA

The DNA eluted by applying the present method to whole blood with solid phase materials of each of examples 2, 4-11, and 13-30 (1 or 2 μL) in either 0.05 M or 0.1 M NaOH was subject to PCR amplification with a pair of 24 base primers which produced a 200 bp amplicon. PCR reaction mixtures contained the components listed in the table below.

| Component | Volume (μL) |
|---|---|
| 10× PCR buffer | 2 |
| Primer 1 (100 ng/μL) | 2 |
| Primer 2 (100 ng/μL) | 2 |
| 2.5 mM dNTPs | 2 |
| 50 mM MgCl₂ | 1.25 |
| Taq DNA polymerase (5U/μL) | 0.25 |
| Template | 1 |
| deionized water | 9.5 |
| Total | 20 |

Negative controls replaced template in the reaction mix with 1 μL of water. A further reaction used 1 μL of template diluted 1:10 in water. Reaction mixtures were subject to 30 cycles of 94° C., 30 s; 60° C., 30 s; 72° C., 30 s. Reaction products analyzed on 1.5% agarose gel showed the expected amplicon.

In separate experiments, the DNA isolated using the particles of example 2 was used in amplification reactions of regions of several different chromosomes as listed below. The results demonstrate that the DNA produced by the isolation procedure is representative of the entire genome.

| Gene | Gene Region | Chromosome |
|---|---|---|
| Factor V Leiden | NA | 1 |
| Corticotropin-β-lipoprotein | exon | 2 |
| precursor | | |
| CFTR (Cystic Fibrosis) | NA | 7 |
| | | (Exon 10) |
| Thyroglobulin | 5′ flanking | 8 |
| Interferon alpha | 3′ untranslated | 9 |
| Factor II (Prothrombin) | NA | 11 |
| Adenosine deaminase | intron | 20 |
| β-2 integrin protein | 3′ untranslated | 21 |

### Example 37

### Capture and Isolation of Nucleic Acid from Whole Blood with Different Amounts of Particles

The DNA from 70 μL of whole human blood was bound onto varying amounts of the particles of example 2 and isolated according to the rapid protocol of example 33. The effect of varying the concentration of NaOH in the eluent was also examined. The amount of DNA eluted was quantified by fluorescence and compared to a standard reference sample of DNA.

| | | | |
|---|---|---|---|
| [NaOH] | 1 mg | 0.5 mg | 0.1 mg |

| | | | | | | | |
|---|---|---|---|---|---|---|---|
| 50 | mM | 1.3 | μg | 1.0 | μg | 0.47 | μg |
| 60 | mM | 1.0 | μg | 1.1 | μg | 0.62 | μg |
| 70 | mM | 1.3 | μg | 1.0 | μg | 0.67 | μg |
| 80 | mM | 1.1 | μg | 0.9 | μg | 0.71 | μg |
| 90 | mM | 0.7 | μg | 1.2 | μg | 0.49 | μg |
| 100 | mM | 1.2 | μg | 1.3 | μg | 0.51 | μg |

### Example 38

### Capture and Isolation of Nucleic Acid from Whole Blood with Different Amounts of Particles

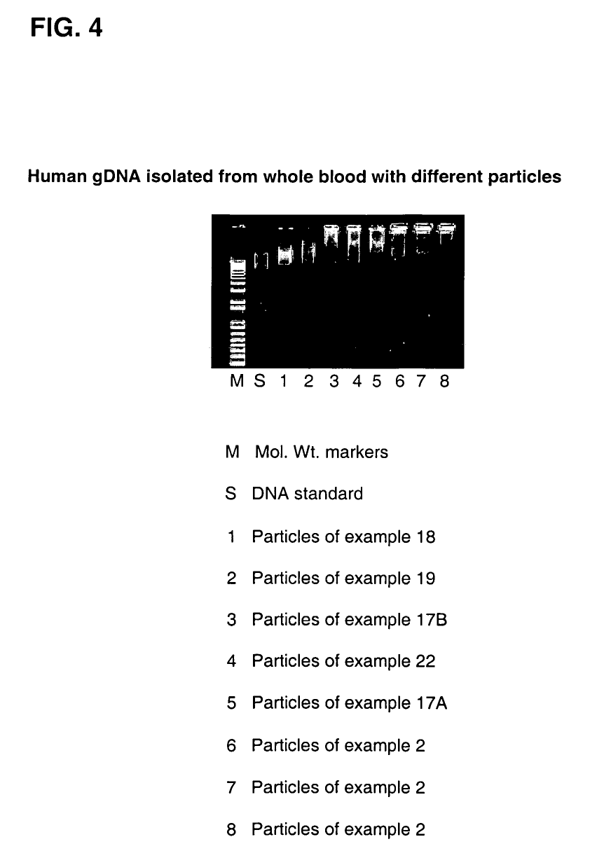

The DNA from 70 μL of whole human blood was bound onto varying amounts of various particles and isolated according to the protocol of examples 31 and 32. The eluents were analyzed by gel electrophoresis as shown in FIG. 4. For comparison, a ladder of size markers ranging in size from 500 bp to 40,000 bp is shown.

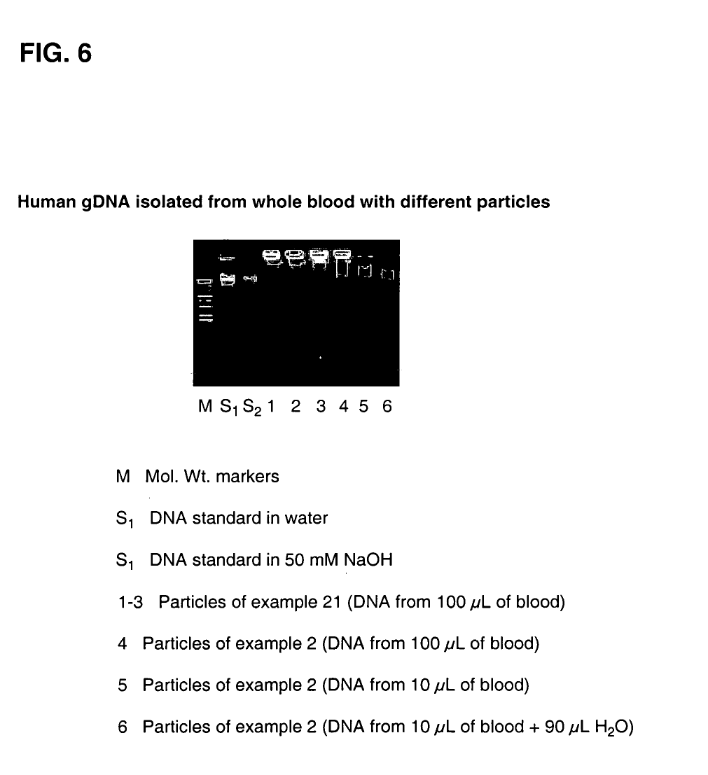

Additional samples are shown in FIG. 6.

### Example 39

### Capture and Isolation of Nucleic Acid from Different Volumes of Whole Blood with Different Amounts of Particles

The DNA from 1 mL of whole human blood was bound onto 5 mg of the particles of example 2 and isolated according to the protocol of examples 31 and 33. Analysis of the eluents (100 μL) of replicate samples by fluorescence indicated yields of 17-24 μg of DNA. Similarly analysis of eluents from the isolation of DNA from 70 μL of blood with 10 mg of the same type of particles eluted with 100 μL of 0.1 M NaOH for 30 s or 1 min, yielded 6.5 μg and 7.3 μg, respectively. Use of the particles of example 5 by protocol 31, 32 yielded 2.8 μg of DNA from 70 μL of blood.

### Example 40

### Capture and Isolation of Nucleic Acid from Whole Blood with Different Solid Phase Materials

The DNA from 100 μL of whole human blood that was bound onto 1 mg of various solid phase materials in accordance with the methods of the invention according to the rapid protocol of example 33 and eluted with 50 μL of 50 mM NaOH solutions. The amount of DNA eluted was quantified by fluorescence and compared to a standard reference sample of DNA.

| Example | DNA (μg) |
|---|---|
| 26 | 1.35 |
| 24 | 0.4 |

The non-magnetic controlled pore glass particles of example 27 (10 mg) and a sintered glass frit of example 28 weighing 10 mg were also tested as described above with the exception that liquids were removed from the solids after centrifugation rather than magnetic separation.

| Example | DNA (μg) |
|---|---|
| 30 | 7.5 |
| 28 | 0.65 |
| 27-A | 11.2 |
| 27-B | 2.63 |
| 27-C | 10.3 |

27-A: 18-50 mesh, 500 Å pore size
27-B: 100-200 mesh, 1000 Å pore size
27-C: 100-200 mesh, 500 Å pore size

1. A method of capturing nucleic acids from a sample of biological or cellular material consisting of:
a) providing a solid phase binding material; and
b) combining the solid phase binding material with a sample of biological or cellular material containing nucleic acids for a time sufficient to bind the nucleic acids to the solid phase binding material.

2. A method of isolating nucleic acids from a sample of biological or cellular material consisting of:
a) providing a solid phase binding material;
b) combining the solid phase binding material with a sample of biological or cellular material containing nucleic acids for a time sufficient to bind the nucleic acids to the solid phase binding material;
c) separating the sample from the solid phase binding material;
d) optionally washing the solid phase binding material; and
e) releasing the bound nucleic acids from the solid phase binding material.

3. The method of claim 1 wherein the biological or cellular material is selected from the group consisting of extracellular nucleic acid, intact cells of animal, plant or bacterial origin and tissue containing intact cells of animal, plant or bacterial origin.

4. The method of claim 2 which is performed in under 5 minutes.

5. A method of capturing nucleic acids from whole blood of an organism consisting of:
a) providing a solid phase binding material; and
b) combining the solid phase binding material with a sample of whole blood for a time sufficient to bind nucleic acids to the solid phase binding material.

6. The method of claim 5 further comprising the steps of:
c) separating the sample from the solid phase binding material;
d) optionally washing the solid phase binding material; and
e) releasing the bound nucleic acids from the solid phase binding material.

7. The method of claim 5 wherein the nucleic acids are contained within leucocytes in the whole blood.

8. The method of claim 6 which is performed in under 5 minutes.

9. The method of claim 1 wherein the nucleic acid is selected from the group consisting of DNA and RNA.

10. The method of claim 1 wherein the nucleic acid is genomic DNA of an organism.

11. The method of claim 1 wherein the solid phase material is selected from silica, glass, sintered glass, controlled pore glass, sintered glass, alumina, zirconia, titania, insoluble synthetic polymers, insoluble polysaccharides, and metallic materials selected from metals, metal oxides, and metal sulfides.

12. The method of claim 1 wherein the solid phase further comprises a magnetically responsive portion.

13. The method of claim 1 wherein the solid phase comprises a covalently linked nucleic acid binding portion.

14. The method of claim 1 wherein the solid phase comprises a non-covalently linked nucleic acid binding portion.

15. The method of claim 1 wherein the solid phase comprises a group selected from the group consisting of hydroxyl, silanol, carboxyl, amino, ammonium, ternary sulfonium groups, quaternary ammonium groups and quaternary phosphonium groups.

16. The method of claim 13 wherein the covalently linked nucleic acid binding portion comprises a quaternary phosphonium group.

17. The method of claim 13 wherein the covalently linked nucleic acid binding portion comprises a carboxyl group.

18. The method of claim 13 wherein the nucleic acid binding portion is attached to the material through a linkage which can be selectively cleaved.

19. The method of claim 1 wherein the bound nucleic acids are released from the solid phase in a strongly alkaline solution.

20. The method of claim 1 wherein the bound nucleic acids are released from the solid phase in a solution which can be used directly in a downstream molecular biology process.

21. The method of claim 19 wherein the bound nucleic acids are released from the solid phase in a solution which can be used directly in a downstream molecular biology process.

22. The method of claim 20 wherein the downstream molecular biology process is a nucleic acid amplification reaction.

23. The method of claim 21 wherein the downstream molecular biology process is a nucleic acid amplification reaction.

24. A method of capturing nucleic acids from a sample of biological or cellular material consisting of:
a) providing a solid phase comprising:
a matrix to which is attached a nucleic acid binding portion;
b) combining the solid phase with a sample or biological or cellular material containing nucleic acids for a time sufficient to bind the nucleic acids to the solid phase.

25. A method of isolating nucleic acids from a sample of biological or cellular material consisting of:
a) providing a solid phase comprising:
a matrix to which is attached a nucleic acid binding portion;
b) combining the solid phase with a sample of biological or cellular material containing nucleic acids for a time sufficient to bind the nucleic acids to the solid phase;
c) separating the sample from the solid phase;
d) optionally washing the solid phase binding material; and
e) releasing the bound nucleic acids from the solid phase.

26. A method of capturing nucleic acids from a sample of biological or cellular material consisting of:
a) providing a solid phase comprising:
a matrix to which is attached, through a selectively cleavable linkage, a nucleic acid binding portion;
b) combining the solid phase with a sample of biological or cellular material containing nucleic acids for a time sufficient to bind the nucleic acids to the solid phase.

27. A method of isolating nucleic acids from a sample of biological or cellular material consisting of:
a) providing a solid phase comprising:
a matrix to which is attached, through a selectively cleavable linkage, a nucleic acid binding portion;
b) combining the solid phase with a sample of biological or cellular material containing nucleic acids for a time sufficient to bind the nucleic acids to the solid phase;
c) separating the sample from the solid phase;
d) optionally washing the solid phase binding material; and
e) releasing the bound nucleic acids from the solid phase by selectively cleaving the linker.

28. The method of claim 24 wherein the solid phase comprises a matrix selected from silica, glass, insoluble synthetic polymers, and insoluble polysaccharides, and an onium group attached on a surface of the matrix selected from a ternary sulfonium group of the formula QR₂⁺X— where R is selected from C₁-C₂₀ alkyl, aralkyl and aryl groups, a quaternary ammonium group of the formula NR₃⁺X— wherein the quaternary onium group wherein R is selected from C₁-C₂₀ alkyl, aralkyl and aryl groups, and a quaternary phosphonium group PR₃⁺X— wherein R is selected from C₁-C₂₀ alkyl, aralkyl and aryl groups, and wherein X is an anion.

29. The method of claim 26 wherein the solid phase comprises a matrix selected from silica, glass, insoluble synthetic polymers, and insoluble polysaccharides, and an onium group attached on a surface of the matrix selected from a ternary sulfonium group of the formula QR₂⁺X— where R is selected from C₁-C₂₀ alkyl, aralkyl and aryl groups, a quaternary ammonium group of the formula NR₃⁺X— wherein the quaternary onium group wherein R is selected from C₁-C₂₀ alkyl, aralkyl and aryl groups, and a quaternary phosphonium group PR₃⁺X— wherein R is selected from C₁-C₂₀ alkyl, aralkyl and aryl groups, and wherein X is an anion.

30. A method of capturing nucleic acids from a sample of biological or cellular material consisting of:
a) providing a particulate binding material; and
b) combining the particulate binding material with a sample of biological or cellular material containing nucleic acids for a time sufficient to bind the nucleic acids to the particulate binding material.

31. The method of claim 30 wherein the nucleic acid is captured in under three minutes.

32. The method of claim 30 wherein the nucleic acid is captured in under thirty seconds.

33. A method of isolating nucleic acids from a sample of biological or cellular material consisting of:
a) providing a particulate binding material;
b) combining the particulate binding material with a sample of biological or cellular material containing nucleic acids for a time sufficient to bind the nucleic acids to the particulate binding material;
c) separating the sample from the particulate binding material;
d) optionally washing the particulate binding material; and
e) releasing the bound nucleic acids from the particulate binding material.

34. The method of claim 33 performed in under five minutes.

35. A method of capturing nucleic acids from a sample of biological or cellular material comprising:
a) providing a particulate binding material; and
b) combining the particulate binding material with a sample of biological or cellular material containing nucleic acids for a time not exceeding three minutes to bind the nucleic acids to the particulate binding material.

36. A method of isolating nucleic acids from a sample of biological or cellular material comprising:
a) providing a particulate binding material;
b) combining the particulate binding material with a sample of biological or cellular material containing nucleic acids for a time sufficient to bind the nucleic acids to the particulate binding material;
c) separating the sample from the particulate binding material;
d) optionally washing the particulate binding material; and
e) releasing the bound nucleic acids from the particulate binding material wherein the method is performed in under five minutes.

37. A kit comprising:
a) a solid phase binding material for capturing nucleic acid directly from biological or cellular material having the ability to capture nucleic acid directly from biological or cellular material without the use of a lysis solution or coating of lysis agent; and
b) and a reagent for releasing nucleic acid from the solid phase.

38. The kit of claim 37 wherein the solid phase binding material is a particulate material.

39. The kit of claim 38 wherein the particulate material is magnetically responsive.

40. The kit of claim 37 wherein the solid phase material is selected from silica, glass, sintered glass, controlled pore glass, sintered glass, alumina, zirconia, titania, insoluble synthetic polymers, insoluble polysaccharides, and metallic materials selected from metals, metal oxides, and metal sulfides.

41. The kit of claim 37 wherein the solid phase comprises a covalently linked nucleic acid binding portion.

42. The kit of claim 41 wherein the nucleic acid binding portion is attached to the material through a linkage which can be selectively cleaved.

43. The kit of claim 41 wherein the reagent for releasing nucleic acid from the solid phase is a strongly alkaline solution.

44. The kit of claim 37 wherein the wherein the solid phase comprises a group selected from the group consisting of hydroxyl, silanol, carboxyl, amino, ammonium, ternary sulfonium groups, quaternary ammonium groups and quaternary phosphonium groups.

45. The kit of claim 41 wherein the covalently linked nucleic acid binding portion comprises a quaternary phosphonium group.

46. The kit of claim 42 wherein the covalently linked nucleic acid binding portion comprises a quaternary phosphonium group and the reagent for releasing nucleic acid from the solid phase is a strongly alkaline solution.
